SEQUENCE LISTING (18 sequences)

SEQ ID NO: 1 - protein, 131 aa, synthetic construct
GVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 2 - DNA, 396 bp, synthetic construct
  source 1..396: mol_type = other DNA
ggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtactaa

SEQ ID NO: 3 - protein, 343 aa, synthetic construct
GVVPQYGGGGNHGGGGNNSGPNMKLKMTVMAAAIAVSSTALANENESNIAQTGFGNDVSVTQLTQGWGNNNSDVTQVGVNNGATVLQQGWRGRNVSDIDQEGMGNNADALQHGTKNSFDLTQSGMFNNSDSQQLGTGNALRVEQEGVKNNATTYQSGWRNYADISQSGSFNNSDVDQEGYLNVAQVNQDGTKLSSDITQSGDYNFADVDQSGSFNDSSIDQNGTQNKAEVLQSGLKNTSDIHQDGKLNIAMVDQSGVNNDASIVQDGRYHQSEVTQSGYNDVAYSSQSGYEHYSSITQSALPGFFGGLASLASNSATHVQMGVNNAAITTQVGGGNTAHVYQH

SEQ ID NO: 4 - DNA, 1032 bp, synthetic construct
  source 1..1032: mol_type = other DNA
ggtgttgttcctcagtacggcggcggcggtaaccacggtggtggcggtaataatagcggcccaaatatgaagttgaagatgacggttatggccgcagctatcgcagtttcatctactgcccttgcaaatgagaatgagtcaaatatcgcccaaactggattcggtaatgacgtctcagttacacaattaacgcaaggttggggcaataataattcagacgttacgcaagttggtgtcaataatggtgcaacagtactccaacaaggttggagaggtcggaatgttagtgacatagaccaagagggtatgggaaataatgccgacgctttacaacacggaactaagaattcattcgacctcactcaaagtggaatgttcaataattctgacagtcaacaacttggtacaggtaatgctttacgtgttgagcaagagggagtcaagaataatgcaacgacataccaaagtggatggcgtaattacgcagacataagtcaaagtggatcattcaataattcagacgtagaccaagaggggtacttgaatgttgcccaagtcaatcaagacggtactaagttaagttctgacatcacgcaaagtggtgactacaatttcgccgacgttgaccaatcgggttctttcaatgactcatctatcgaccaaaatggtacgcaaaataaggcagaggtattgcaatcgggtctcaagaatacgagtgacatacaccaagacggtaagcttaatatagccatggtagaccaatctggggttaataatgacgccagtatcgtccaagacggacgttaccaccaatcagaggtcactcaatctggatacaatgacgtcgcatactcatctcaaagtggttacgagcactactcctctatcacgcaatcagcattaccaggtttcttcggtgggttggctagtttggcatccaatagtgcaactcacgtccaaatgggagttaataatgccgctatcacaacgcaagttggtggtggaaatacagctcacgtataccaacactaa

SEQ ID NO: 5 - protein, 505 aa, synthetic construct
GVVPQYGGGGNHGGGGNNSGPNMKMKKTKIASALLLVAATGWASAQEIPASKSVDFNASADFVAAAEGNEITLTQTAPEGNEVGNESVLSQEGDLNAIVVEVTGDANEVLASQMQSGNTFFASVTGNGNFLESEQDSLDSTAEFVVEGDDNLVSLVQLGDGGPFGFIFSRSINSIAGSGNTLSVYQGDGGNWANNAIAGNDNTVFVDQSGDWHESYVRELSGDANLIDVIQDGFYNISDLTVVGSDNEVEVDQDGDENLITWNMVGDGNVLEFEQDGDGNEITTGVFEGSDNEVNIDQIGDVNLATVETIGGGVNTFEINQVGEGNVAYAGVIGLFNEFDLTQVGDANEISTVNFDGWFNTVEIEQDGDANLALAQAGIGDAAYVADANLIEMSQEGNENIASVELARDITSSGNEIMVAQTGELNLLDLLVNGNDNVISVMQEGAGNWVTDDMGGQFVISGDMNTFEVTQMGNDNLVTGSITGNGGTVSVTQVGDYNVATVVQM

SEQ ID NO: 6 - DNA, 1518 bp, synthetic construct
  source 1..1518: mol_type = other DNA
ggtgttgttcctcagtacggcggcggcggtaaccacggtggtggcggtaataatagcggcccaaatatgaagatgaagaagacaaagatcgcaagtgcactcttgttagtcgctgctactggttgggcaagtgcacaagagatacctgcctcaaagtccgtcgacttcaatgcttccgcagacttcgtcgccgctgccgagggtaatgagatcacattaactcaaactgctcccgagggaaatgaggtcggtaatgagagtgttttatcccaagagggagacttaaatgccatcgttgtcgaggtcacgggtgacgccaatgaggtattagcaagtcaaatgcaatctggtaatactttcttcgccagtgtaactgggaatgggaatttcttagagagtgagcaagactcgttagacagtacggcagagttcgttgtagagggagacgacaatttggtttccttagttcaattaggtgacggtggtcccttcggtttcatcttctcccgttcgatcaattccatcgctggtagtgggaatacgttatcagtataccaaggtgacggtggtaattgggctaataatgctatagctggaaatgacaatacagtcttcgtagaccaaagtggagactggcacgagtcgtacgttcgtgagttgtccggtgacgcaaatttgatcgacgttatccaagacgggttctacaatatctcagaccttacagtagttggatctgacaatgaggtcgaggtcgaccaagacggtgacgagaatctcatcacatggaatatggttggtgacgggaatgtattggagttcgagcaagacggagacggtaatgagatcacgactggtgtcttcgaggggtccgacaatgaggttaatatagaccaaataggggacgttaatttagctactgttgagacaatcggtggtggggtcaatactttcgagatcaatcaagtaggtgagggtaatgtagcatacgcaggagtaatagggctcttcaatgagttcgacttgacgcaagttggagacgccaatgagatcagtacagttaatttcgacgggtggttcaatactgttgagatcgagcaagacggtgacgctaatcttgccttagcccaagcagggatcggtgacgcagcctacgttgccgacgcaaatttaatcgagatgtctcaagagggtaatgagaatatcgcaagtgtagagttagctcgtgacatcacatcctcaggtaatgagatcatggttgctcaaacaggtgagttaaatttattagacttgcttgttaatggaaatgacaatgtaatcagtgtcatgcaagaaggggccggaaattgggttacggacgacatgggtggtcaattcgtcatatccggagacatgaatacattcgaggtaactcaaatggggaatgacaatcttgttactggtagtataacgggtaatggtggtacagtaagtgtcacacaagtcggtgactacaatgtcgccacagtagttcaaatgtaa

SEQ ID NO: 7 - protein, 945 aa, synthetic construct
GVVPQYGGGGNHGGGGNNSGPNMKTSIYTSVSALALMIGMPAVAQTNTSTVDQTGAAAAATVNQTGSNNTSDIDQNGNGLAYTTGPNTGRTVVADVEQTGSNGFSQVTQQGGRSSATVDQGGTGMRSTITQGNASTDLGNTATVIQNGTGGGVGTGSTITQTGGTGQAYVNQGAATNGAISTITQTGNNQRASVFQTSGAADSSVSQSGGAANVFVSQDGSSESDITQTGGNSEASVRQIGDGNTSLIEQTGVNGDVGDPDNNLANQDTNIGVSQTGDNNSSTVRQTGNDQVADVIQTGDFNTASITQDGNFADASVTQTGNNNTGTVIRQDGDGSGDNDPASLPVIPTTNATADISQTGDFNEASISQAAVPVEATITQLGDSNDSSIVQSSTASGAKATNLQTSDNNLSTITQDSDAEASVTQGGSQFNPGPFAGRDNNVSTVAQGTGAAGSLATVNQDGVLNLSDINQNAANSTASVTQTNIFNTSSVIQSGSGGATADVTQGGGYGDNVSNITQTTNASATVVQTGQPSVGPDFTNISDIVQNGTGGIASVTQNGSNNESDVAQGGTDGDATVDQDGTFLRSTITQTGGEATALVTQTGNANVSTISQSAASAGADATVTQSGGENRSDVNQTGAAKAVVTQAGTEVGYNLAPPNNDSFVVQSADGADANVSQTGELNLSNVNQSGGASEADVTQTGVYNRSTVTQTVGGAKATVTQSGDPANGGPGVGQGDNLSIVSQSGASTATVTQTANVPTNGFPSNDSFVSQTGDDSDATVDQTGNDNASDVFQGSDAIADVTQDGTNNTSTVTQSAATSAFVEQIGARNTSTVTQSGGSTVAPYAAYVQQLGNDGLSVVEQDGANNEATLTQSAGSSFADSSIDQSGNNNLADVTQGGFDNTSTIVQSGNNNAATVNQSSTGNVSDIIQSGNGNSATVTQGGGVL

SEQ ID NO: 8 - DNA, 2913 bp, synthetic construct
  source 1..2913: mol_type = other DNA
ggtgttgttcctcagtacggcggcggcggtaaccacggtggtggcggtaataatagcggcccaaatatgaagacttcaatctacacatctgtctccgccttggccttaatgataggtatgcccgcagtcgctcaaacgaatacatccactgttgaccaaacaggtgctgccgcagcagccacagttaatcaaacaggttcaaataatacatctgacatcgaccaaaatggaaatggtcttgcatacactacaggtcccaatacaggtcgtacggtagtagccgacgtcgagcaaacagggtcgaatggattctcccaagtaacgcaacaaggcggaagaagttctgcaactgtagaccaaggtggtactggtatgcggtctacaatcacacaaggaaatgcatctactgaccttgggaatacggctacggtcatccaaaatgggacgggtggtggtgtcggtacaggatctacgatcacacaaactggtggaactggtcaagcttacgtaaatcaaggggcagctacgaatggagcaatcagtacgatcacacaaactggtaataatcaacgggcttcggtcttccaaacgtcaggggcagctgactcctctgtttcccaatcaggtggggcagcaaatgttttcgtatcccaagacggtagttcggagtcagacatcacacaaacaggtggtaattcagaggcttcagtccgtcaaatcggtgacggaaatacttctctcatagagcaaacgggagtcaatggtgacgttggagacccagacaataatttggcaaatcaagacactaatataggtgtttcccaaacaggtgacaataatagttctacggttcgtcaaacaggaaatgaccaagtcgctgacgtaatacaaacgggtgacttcaatacagcttcaataacacaagacggaaatttcgccgacgcaagtgtcactcaaactggtaataataatacgggtacagttatccggcaagacggagacggttctggggacaatgaccctgcatcacttcctgttataccaacaacaaatgcaacggccgacatcagtcaaacgggtgacttcaatgaggcctctatatcccaagcagccgtacccgttgaggcaacaatcacacaacttggagactccaatgacagtagtatcgtccaatcatctactgcctcaggtgctaaggcaactaatcttcaaacgtctgacaataatttatctacgataacgcaagacagtgacgcagaggcatcggtcacgcaaggtggttctcaattcaatcccgggccattcgccgggcgggacaataatgtatctacggtagcccaaggaacgggtgcagccggttcattggcaacagtaaatcaagacggggtcttaaatttatcagacataaatcagaatgctgcaaattctacggcctcggtaactcaaacaaatatattcaatacatcgagtgtaatccaatctgggtctggtggtgccactgcagacgttacgcaagggggggggtacggagacaatgtttccaatatcactcaaacaacgaatgcctcggccactgtcgtccaaacgggtcaaccatctgtcggaccagacttcacgaatatctcggacatcgtacaaaatggtactggcgggatagcatcagttactcaaaatggatctaataatgagtcagacgttgcccaaggtggtacagacggggacgctacggtagaccaagacgggacatttctccgttccactatcacacaaactggcggagaggctacggccttggttactcaaacgggtaatgctaatgttagtacgatcagtcaatcggcagccagtgctggagctgacgccactgttactcaatcaggcggagagaatagatcggacgtaaatcaaacaggagcagctaaggcagtagttacgcaagctgggactgaggtcggttacaatcttgcacccccgaataatgactccttcgtcgttcaatcagccgacggagcagacgccaatgtctctcaaacgggtgagttaaatctttcgaatgttaatcaaagtggcggagcgtccgaggctgacgtaacacaaacaggtgtatacaatcgttcgacagttacacaaacagtaggtggtgccaaggccacggttacacaatcgggtgaccctgctaatggtggaccaggggtaggacaaggtgacaatttatccatcgtcagtcaatctggtgcttctacggctacagttactcaaactgctaatgttccaacgaatggattcccatccaatgactccttcgtttcgcaaacgggagacgacagtgacgctacagtagaccaaacgggaaatgacaatgcaagtgacgtattccaaggtagtgacgccatcgctgacgtaacgcaagacggtactaataatacttctacggtcacgcaatctgcagcaacttcggccttcgttgagcaaataggggcccgtaatacgagtactgtcactcaatctggtggttctacagttgcgccttacgcagcttacgttcaacaactcggtaatgacggtcttagtgttgttgagcaagacggggccaataatgaggcaacacttacacaaagtgccgggtcgtcgttcgctgactcgtctatcgaccaaagtggtaataataatcttgccgacgtaactcaaggtggattcgacaatacaagtacaatcgtccaaagtgggaataataatgctgccactgttaatcaaagtagtacagggaatgtctcagacataatacaatctgggaatggaaattcagcaactgtcacacaaggtggtggggtacttgaaaacctgtattttcagggcggctctggtggctctggtggctctggcggcagcgggcatcaccaccaccatcattaa

SEQ ID NO: 9 - protein, 1087 aa, synthetic construct
GVVPQYGGGGNHGGGGNNSGPNMRLWARQTKRSSSAGVLGEILMKKTALLGVSIIALSAAAPAFGQSQSTVTQTGNNSTVDVTQGGPDGGNVSTVDQSASDSNVTINQEGFDDNLSGVTNTSTVTQAGLNQDVQSTQVGDDQTSSVIQSGVDMEALLVQGGAGNSSTINQSDTDNFADVIQDGDDNISNVSQSDEDGTVDVDQLGERLTSTISQQDDNNQNAVVLQTNADNTSTITQRASNSDVFVTQSGAENTSTVLQGNISDNQEATILQTGDRNSSDVQQGFDLSGPLDDDNVTFVTQNGDDNTAVVRQINERNLADILQEGDRNEARVVNQGTGVSGGGNEDNIADIDQFGDDNFASINQPGEDGTFTVVQNGFDSSSTGLQSGSNDTASVIQNGEFDSSNVEQSGSGNNATVTQEGFDLDGSLLANSSTVLQSGTGDTATVSQVGDNQSSFVAQSGSVSTASVVQGGVGNSSGITQSGTDVTATVNQEGDGNSSTVTQANSLSDAVVSQTGDFDTSIVAQSGIDEQANVDQAGETGVSEVTQSGDSNFADVFQRGAENTSFITQSGLDGNVDLDQTGDENFSNAIQAGFEGDIIVNQTGDGNSSIVNQQGDATGFNFATIDQSGNGNTSEAIQFNNSNNANILQSGDDNTSFATQTGTGVIADVDQIGEGATSTVTQDGSGNLAIVSQEGIDFDLSGTTDTSLVAQTGTGQNAEVTQVGDGNSSSVVQNNADNTASVLTAGIGNSSFVTQNGLTGLATVDQQGDGNSSIVSQGGSNASADVSQQSDTSSSNVNQAGDDVLALVDQAGANHSASIVQLTTGPTGGVDLNNGALIDQQGDGNTANITQSGGIGVFFNVLSGTGLSSGQIAEISQNGSGNTGTVAQSGTTNIGRFFQDGDDNSATISQTGQDSDSSFGQTGNNNTANVTQALIAPPGGIAFADPNQNGDGNSATVVQNGTVTGFFTTRAQSAQIGNGNTLTTTQSGTDDIVFIDQIGDGNMSMVNQLAGGVSNEGDINQTGDDGISELTQSGTDQFAELFQSGDLNTSLITQAGVTNTATVTQGSDGNFSSVNQNGTGNSTTVTQ

SEQ ID NO: 10 - DNA, 3339 bp, synthetic construct
  source 1..3339: mol_type = other DNA
ggtgttgttcctcagtacggcggcggcggtaaccacggtggtggcggtaataatagcggcccaaatatgcggctttgggcccgtcaaactaagcgttcttcttcagcaggtgtcttaggtgagatattaatgaagaagactgccttgttgggtgtttccataatagctctttccgccgcagctcccgcattcggacaatcacaatccactgtcacacaaacaggaaataattcgacggttgacgtaacgcaaggcggtcccgacggtgggaatgtatccactgtagaccaatcagcttccgacagtaatgttactatcaatcaagaggggttcgacgacaatctttcaggagttactaatacttcaactgtcacgcaagccggacttaatcaagacgttcaatcaacacaagttggggacgaccaaacttcgtcggtaatccaatctggagtcgacatggaagcacttctcgttcaaggcggagccggaaattcttccactatcaatcaatccgacacagacaatttcgccgacgtcatacaagacggtgacgacaatatatctaatgtttcgcaatcggacgaggacggaactgttgacgtcgaccaattaggtgagcgacttacatctactatatcacaacaagacgacaataatcaaaatgccgttgtactccaaacaaatgcagacaatacatccacaataacacaacgtgcttccaattcagacgtattcgttacacaatccggagcagagaatacttccactgtacttcaagggaatatatccgacaatcaagaggcaactatattacaaactggtgaccgtaattctagtgacgtccaacaaggattcgacctttcgggacctttggacgacgacaatgtcacgttcgtcacgcaaaatggtgacgacaatactgccgttgttcgacaaataaatgagcggaatttagccgacatcttacaagagggagaccgtaatgaggcacgtgtcgtcaatcaaggaacaggggtttcaggggggggtaatgaggacaatatagccgacatcgaccaattcggggacgacaatttcgcaagtatcaatcaacctggtgaggacgggactttcacggtagttcaaaatggattcgactcctcatcaacaggtttacaatccggttccaatgacactgcttccgtcatccaaaatggggagttcgactcttcaaatgtcgagcaatccggatctgggaataatgctactgtcacacaagagggtttcgacttagacggttcacttcttgctaattcctctactgtcttacaaagtggtacgggtgacacggccacagtaagtcaagttggtgacaatcaatcgtccttcgtagcacaatcgggttcggtatctacggcctcagttgtccaaggtggggtcggtaattcttctggtatcactcaaagtggtacggacgtaacagccacagttaatcaagagggtgacgggaattcctctactgtcacacaagctaattcattatcggacgctgtagtttctcaaacgggagacttcgacacaagtatcgttgcacaaagtggaatagacgagcaagcaaatgttgaccaagcaggtgagacaggagtctccgaggtaacgcaaagtggtgactccaatttcgccgacgtcttccaacgtggagccgagaatacgagtttcataacacaatccggtttggacgggaatgtcgacttggaccaaacaggagacgagaatttctccaatgcaatacaagctggtttcgagggtgacatcatagtcaatcaaactggggacggtaattcttcaatagttaatcaacaaggggacgctactggtttcaatttcgctaccatagaccaatcagggaatggtaatacctctgaggcaatccaattcaataatagtaataatgctaatatcttgcaatcaggtgacgacaatacgtcattcgccacgcaaacgggtactggggttatagccgacgtcgaccaaatcggagaaggggccacatctacggtcacacaagacggaagtggaaatcttgcaatcgtttcacaagagggtatcgacttcgacctttcaggaactacggacacttcacttgttgcccaaacgggaactggtcaaaatgccgaggtcacacaagttggagacggtaattcctcatctgtagtacaaaataatgctgacaatacggctagtgttttaacagctggtatcgggaattcatccttcgttacacaaaatgggcttacaggtcttgccactgttgaccaacaaggagacggtaattcatccatcgtttcacaaggtggttctaatgccagcgcagacgtaagtcaacaatccgacactagttcctcaaatgttaatcaagctggtgacgacgtcttagccctcgtcgaccaagcaggtgcaaatcactcagcatctatcgtccaattgacaacaggacctacgggtggtgtagacttaaataatggggcattaatagaccaacaaggtgacggaaatactgctaatatcacacaaagtggtggtatcggtgtcttcttcaatgtattgagtggaactgggctttcttcaggtcaaatcgccgagataagtcaaaatggttcaggtaatacaggtacggtagcccaatctgggacgacgaatataggacgattcttccaagacggagacgacaattccgccactatctcccaaactggacaagactcggactcgtctttcggacaaacaggaaataataatacggctaatgtcactcaagcacttatcgctccgcccggcggaatcgcattcgcagacccaaatcaaaatggtgacgggaattcagctactgtagttcaaaatggaacggtcacaggattcttcacgacacgtgctcaatccgcccaaataggtaatggtaatactctcacgacgacacaatctggtactgacgacatagttttcatcgaccaaatcggtgacggaaatatgtccatggtaaatcaacttgctggcggagttagtaatgaaggggacataaatcaaactggagacgacgggatctcagagcttacgcaatccggtacggaccaattcgccgagctcttccaaagtggtgaccttaatacatcgttaataacgcaagctggggtcacaaatacagccactgtcacacaaggttccgacggtaatttctcttctgttaatcaaaatggaacagggaatagtacgactgtcacgcaagaaaacctgtattttcagggcggctctggtggctctggtggctctggcggcagcgggcatcaccaccaccatcattga

SEQ ID NO: 11 - protein, 1196 aa, synthetic construct
GVVPQYGGGGNHGGGGNNSGPNMKFSARKTLLLTGVAVLAMSAAASAQEANTGYIGQRDDSNFAYLSQEGANRGFIYQHGQQNVFLPLAAGGAAGDAQQGKNDLGVMQLGSKNVVGGDFLQKGENVGGIAQFGTNNLVDKFRQDGANRGTIFQGGNANVARNTQEEGDSGNDTGIIQLNDSPNKPGNYAESNQLGANGGNFSGILQNGSGNAAINNQTDETSSNAVTAQSGTGNAAINNQTASIDGNAGILQVGKGNGAINTQTGSSLDATAIIAQNGKDNSAVNTQTNVDPTSATVIQDGNNNAAANVQTSVNNSNAFILQDGNNNTASNLQSPGTQAGPADSLNASIAQSGNKNNAVNLQAGGSGGGKKNTAQTVQDGNRNTALNSQIGDYHNNGYNNDNKAVIGQVGNDNLAVNTQNDGRNSTATILQGGDRNDAYNLSEGGTNHSSAIAQFGDDNLAVNARNRGGNPLEGNDAAILQFGDDNIAANYQNDAIGSEQFTNNSANIGQFGSGNIATNFQDTGSDNEGNITQFGTNNGASNAQVGGGVENTANILQIGSGNSGANVQSGGTEATANIVQGGSNNVAANLQTNVDPTNASILQLGSRNTAGNVQADLVNSDALIVQDGARNEASNLQTDGTNMATVIVQKGDRNNAGTIQAGSTDVDAFTLQNGDRNEASNGQSGVTGGTAGIIQDGNRNVAGSVQLTSSDVDASIIQSGNRNDAANLQSGVTGGSAGILQDGRDNTAANLQLSSTGVDATIIQSGRFNEAVNSQTASSLSNAFIIQGAGGNNESANVQDDVSGATALIGQFGWGNTAGNLQQADSSNVIAGILQVGSGNTAYNTQGATQTATYEVLPAFNLTTTASVPGLSNYLTPNMQGPFIFGIAGGPTTLTGSGSAPAEDSIALVAQFGNDNSAANVQIGQLIAPASGIASITTTTYGINTSVQNGGASWLLGPKTITHSQAQSTQTTSTPFTNPLGEINVPTVASAAGIVQVGDDNDAFNFQTVAAGSLAATVQIGDGNGAANVQTATLGAAAAIVQSGNGNTASNLQNGAILSAAGIIQAGHDNVATTTQIASLSNAALTIQNGKKNVAGTNQSNTVGNTSLIVQSSNNSLALVDQTGAGGHSSAVVQAGAYSNAWISQTGSASNSVVLQYGSGSSDADRNYAIVSQGNSGQNSVVIQAGRANVAFVSQN

SEQ ID NO: 12 - DNA, 3591 bp, synthetic construct
  source 1..3591: mol_type = other DNA
ggtgttgttcctcagtacggcggcggcggtaaccacggtggtggcggtaataatagcggcccaaatatgaaatttagtgcgcgcaaaacgctgcttctgactggggtcgctgtattagccatgtctgccgctgcgtcagctcaggaggcaaacacgggctatatcggacaacgtgatgactcaaactttgcgtatctgagccaagaaggtgcaaaccgcggtttcatttatcaacatgggcaacagaacgtatttttgccactggctgccggaggcgcagcaggcgatgcccagcaagggaaaaacgacttaggcgtaatgcaattaggtagtaaaaatgttgtaggcggggatttcttgcaaaagggagagaatgttggaggtattgctcaattcgggaccaacaacctggtggacaaatttcgtcaagacggagcgaatcgcggaacaattttccaaggcggcaacgcaaacgtcgcacgcaatacgcaggaggagggtgattccggtaatgacactggcattatccagctgaacgactctccaaataaaccagggaattacgctgaatccaatcagctgggcgcaaacggtggcaatttctcaggcattcttcaaaacggttccggtaacgcagcgattaataatcagaccgacgagacaagttcgaatgcagtgaccgcccagagtggaacagggaacgctgccattaataatcaaactgcctccatcgacggtaatgccggtattttacaggttggtaagggcaacggcgcaatcaacactcagacggggtcaagtcttgatgcaacggcaatcattgctcagaatggaaaagataactccgcggtgaacacgcagactaacgtagatccgacaagtgcaacagtgatccaagatgggaataacaatgcagctgcaaacgttcagacgtcagtgaacaattcaaatgcgttcattctgcaagatggtaacaacaatacagcaagcaaccttcaatctcctggcacacaggccggtcccgcggatagcttaaatgcgagtatcgcgcagagcggaaacaaaaataatgcagtcaacctgcaagctggtggctcaggaggtgggaaaaagaacacagcacaaacagtacaggacggtaaccgcaatacggcactgaactcacaaatcggagattaccacaacaacggttacaataacgataacaaagctgtcattgggcaggttggaaatgacaatctagctgtaaatacacaaaacgatggaaggaacagcaccgctacgattttgcaaggtggcgatcgtaacgacgcgtataacctgagcgagggtggcacgaaccacagcagcgcgatagcgcagtttggtgacgacaacctggcagtgaacgcgcgtaatcgcggtggtaacccgctggaaggtaacgacgcagccattctccaattcggcgacgataacatcgcagcgaactaccagaacgacgcgattggtagcgagcagttcaccaacaactctgccaacatcggccaattcggtagcggtaacatcgcaaccaacttccaggacaccggtagcgataacgagggcaacatcacccagttcggtacgaacaatggcgcgtccaatgcgcaagttggtggcggtgtggaaaataccgctaatatcctgcagattggttccggtaatagcggtgcgaatgtgcaatctggcggtacggaagcgaccgcgaatatcgtgcaaggtgggagcaataacgtagccgcgaaccttcaaacaaacgtggatccgaccaatgcgtcgatcttgcagctgggttctcgtaataccgccggtaacgtccaggcggatctggttaattctgacgcactgattgtgcaggacggtgcacgtaacgaggcgagcaatttgcagaccgatggcaccaacatggctacggtcatcgtgcaaaaaggtgatcgtaacaacgccggcaccattcaagcaggcagcaccgacgtggacgcttttactctgcaaaacggtgacagaaatgaggctagcaacggtcaaagcggcgttaccggcggtactgcgggtatcattcaggatggtaaccgcaacgtcgctggttcagttcagttgaccagcagcgatgttgatgcgtccattatccaaagcggaaatcgtaacgatgccgcaaacctgcagtccggcgttaccggcggctctgcgggcattttacaagacggccgcgataataccgcggcaaacctgcagctgagctcgactggcgttgacgcgaccatcatccagagcggccgttttaatgaagccgttaattctcagactgcctcctcactgtccaatgcttttatcattcagggcgctggcgggaacaatgaaagcgctaacgtgcaagacgacgtgtcaggggcgactgcattaatcgggcaatttggttggggaaataccgccggcaatttgcaacaggcggattcctctaatgttatcgcgggaatcctgcaggtgggttcgggcaataccgcgtacaatacgcaaggggccacgcaaactgcaacgtatgaggtccttcctgcctttaatttaacgacgaccgcctccgtgcccggtttatccaactatttgacgccgaacatgcaaggccctttcatctttggaattgcgggtggaccgacaaccttgaccgggtccggctctgctcccgcagaggacagtatcgccctggtagcacaattcgggaatgataactctgctgccaatgtgcaaatcggccagttaatcgcaccggctagtgggatcgcatccatcacaactacaacttatggtattaataccagtgtgcagaacggaggggcgtcttggcttcttggacctaagaccatcactcactcccaagcacaaagcacacaaactacctccactccgttcactaaccccttgggcgaaattaatgtacctaccgttgcttccgcggccgggatcgtacaagttggcgacgacaatgatgcattcaatttccagaccgtggcggcgggtagtctggcggccactgttcagattggtgatggcaatggtgccgcgaacgtacaaacggcaacgttaggggctgcagccgcaattgtgcagtcagggaatggaaatacagcatcaaacctgcagaacggcgccattctttcagcagcaggcattattcaagcagggcatgataacgtcgcgactacaacccagattgccagcttgagtaacgccgccttgaccattcagaacggtaagaagaatgtggcaggcaccaatcagagcaatacggtcggaaatacaagtctgattgtccagtcttccaataattcgcttgcacttgtcgatcaaacaggagcggggggtcactcaagcgccgtggttcaagctggcgcatacagtaatgcatggatcagtcagaccgggtcggcgtcgaactcagtggttcttcaatacggcagtggctcaagcgacgcggatcgcaactatgctatcgtctcgcaagggaactcgggccagaactccgtagtgattcaggctgggcgtgctaatgtggcattcgtgagtcagaactaa

SEQ ID NO: 13 - protein, 387 aa, synthetic construct
GVVPQYGGGGNHGGGGNNSGPNMKLKMTVMAAAIAVSSTALANENESNIAQTGFGNDVSVTQLTQGWGNNNSDVTQVGVNNGATVLQQGWRGRNVSDIDQEGMGNNADALQHGTKNSFDLTQSGMFNNSDSQQLGTGNALRVEQEGVKNNATTYQSGWRNYADISQSGSFNNSDVDQEGYLNVAQVNQDGTKLSSDITQSGDYNFADVDQSGSFNDSSIDQNGTQNKAEVLQSGLKNTSDIHQDGKLNIAMVDQSGVNNDASIVQDGRYHQSEVTQSGYNDVAYSSQSGYEHYSSITQSALPGFFGGLASLASNSATHVQMGVNNAAITTQVGGGNTAHVYQHGGSGSSGSGGSGGGSGSSGSGGSGGGSGSSGSGGSGRHRHRHRH

SEQ ID NO: 14 - DNA, 1164 bp, synthetic construct
  source 1..1164: mol_type = other DNA
ggtgttgttcctcagtacggcggcggcggtaaccacggtggtggcggtaataatagcggcccaaatatgaagttgaagatgacggttatggccgcagctatcgcagtttcatctactgcccttgcaaatgagaatgagtcaaatatcgcccaaactggattcggtaatgacgtctcagttacacaattaacgcaaggttggggcaataataattcagacgttacgcaagttggtgtcaataatggtgcaacagtactccaacaaggttggagaggtcggaatgttagtgacatagaccaagagggtatgggaaataatgccgacgctttacaacacggaactaagaattcattcgacctcactcaaagtggaatgttcaataattctgacagtcaacaacttggtacaggtaatgctttacgtgttgagcaagagggagtcaagaataatgcaacgacataccaaagtggatggcgtaattacgcagacataagtcaaagtggatcattcaataattcagacgtagaccaagaggggtacttgaatgttgcccaagtcaatcaagacggtactaagttaagttctgacatcacgcaaagtggtgactacaatttcgccgacgttgaccaatcgggttctttcaatgactcatctatcgaccaaaatggtacgcaaaataaggcagaggtattgcaatcgggtctcaagaatacgagtgacatacaccaagacggtaagcttaatatagccatggtagaccaatctggggttaataatgacgccagtatcgtccaagacggacgttaccaccaatcagaggtcactcaatctggatacaatgacgtcgcatactcatctcaaagtggttacgagcactactcctctatcacgcaatcagcattaccaggtttcttcggtgggttggctagtttggcatccaatagtgcaactcacgtccaaatgggagttaataatgccgctatcacaacgcaagttggtggtggaaatacagctcacgtataccaacacggtggatctggtagcagcggctctggtggttctgggggcggaagtggctcctctgggagcggggggtcgggtggtggctcgggttcatctggtagtggcggttcgggtcgccatcgccatcgccatcgccattaa

SEQ ID NO: 15 - protein, 564 aa, synthetic construct
GVVPQYGGGGNHGGGGNNSGPNMKLKMTVMAAAIAVSSTALANENESNIAQTGFGNDVSVTQLTQGWGNNNSDVTQVGVNNGATVLQQGWRGRNVSDIDQEGMGNNADALQHGTKNSFDLTQSGMFNNSDSQQLGTGNALRVEQEGVKNNATTYQSGWRNYADISQSGSFNNSDVDQEGYLNVAQVNQDGTKLSSDITQSGDYNFADVDQSGSFNDSSIDQNGTQNKAEVLQSGLKNTSDIHQDGKLNIAMVDQSGVNNDASIVQDGRYHQSEVTQSGYNDVAYSSQSGYEHYSSITQSALPGFFGGLASLASNSATHVQMGVNNAAITTQVGGGNTAHVYQHGGSGSSGSGGSGGGSGSSGSGGSGGGSGSSGSGGSGADNKFNKEQQNAFYEILHLPNLNEEQRNGFIQSLKDDPSQSANLLAEAKKLNDAQAPKAGGGGADNKFNKEQQNAFYEILHLPNLNEEQRNGFIQSLKDDPSQSANLLAEAKKLNDAQAPKAGGGGADNKFNKEQQNAFYEILHLPNLNEEQRNGFIQSLKDDPSQSANLLAEAKKLNDAQAPKA

SEQ ID NO: 16 - DNA, 1695 bp, synthetic construct
  source 1..1695: mol_type = other DNA
ggtgttgttcctcagtacggcggcggcggtaaccacggtggtggcggtaataatagcggcccaaatatgaagttgaagatgacggttatggccgcagctatcgcagtttcatctactgcccttgcaaatgagaatgagtcaaatatcgcccaaactggattcggtaatgacgtctcagttacacaattaacgcaaggttggggcaataataattcagacgttacgcaagttggtgtcaataatggtgcaacagtactccaacaaggttggagaggtcggaatgttagtgacatagaccaagagggtatgggaaataatgccgacgctttacaacacggaactaagaattcattcgacctcactcaaagtggaatgttcaataattctgacagtcaacaacttggtacaggtaatgctttacgtgttgagcaagagggagtcaagaataatgcaacgacataccaaagtggatggcgtaattacgcagacataagtcaaagtggatcattcaataattcagacgtagaccaagaggggtacttgaatgttgcccaagtcaatcaagacggtactaagttaagttctgacatcacgcaaagtggtgactacaatttcgccgacgttgaccaatcgggttctttcaatgactcatctatcgaccaaaatggtacgcaaaataaggcagaggtattgcaatcgggtctcaagaatacgagtgacatacaccaagacggtaagcttaatatagccatggtagaccaatctggggttaataatgacgccagtatcgtccaagacggacgttaccaccaatcagaggtcactcaatctggatacaatgacgtcgcatactcatctcaaagtggttacgagcactactcctctatcacgcaatcagcattaccaggtttcttcggtgggttggctagtttggcatccaatagtgcaactcacgtccaaatgggagttaataatgccgctatcacaacgcaagttggtggtggaaatacagctcacgtataccaacacggtggttctggctcgtctggatcggggggaagcggtggtggttcgggctcatctggctctggcggatcagggggtggtagtggatcttcaggatctggtggatccggcgcggataataaattcaataaagagcagcagaacgccttttacgagattcttcacttgccgaatcttaatgaggaacaacgtaacgggtttattcaatcactgaaagatgaccccagccagtcggcaaatcttcttgccgaagcgaagaaacttaacgacgcacaggcccctaaggcggggggtggcggggctgataataagtttaacaaggaacaacaaaacgctttttatgagatcttgcacttaccaaacttgaatgaggagcagcgtaatgggttcattcagagcttgaaggatgatcctagccaaagcgctaacttgttggcagaggcgaaaaagttaaacgacgcacaagccccaaaggcgggcggagggggagctgacaacaagttcaacaaagagcagcaaaatgccttctatgaaattctgcatttgccgaatttaaatgaagaacagcgcaatggttttatccaatcgttaaaagacgatccatcgcaatccgccaaccttctggcggaagcgaagaagctgaatgatgcacaagcaccgaaagcgtaa

SEQ ID NO: 17 - protein, 20 aa, synthetic construct
MKLLKVAAIAAIVFSGSALA

SEQ ID NO: 18 - protein, 22 aa, synthetic construct
GVVPQYGGGGNHGGGGNNSGPN

(19) United States
(12) Patent Application Publication
(10) Pub. No.: US 2026/0042805 A1
(43) Pub. Date: Feb. 12, 2026

(54) PRODUCTION OF CSGA-LIKE FUNCTIONAL AMYLOIDS FOR ENGINEERED LIVING MATERIALS

(71) Applicants: VIRGINIA TECH INTELLECTUAL PROPERTIES, INC., Blacksburg, VA (US); Northeastern University, Boston, MA (US)

(72) Inventors: Anna Duraj-Thatte, Blackburg, VA (US); Hoda Hammad, Blackburg, VA (US); Avinash Manjula-Basavanna, Boston, MA (US)

(21) Appl. No.: 19/293,006

(22) Filed: Aug. 7, 2025

Related U.S. Application Data
(60) Provisional application No. 63/680,451, filed on Aug. 7, 2024.

(51) Int. Cl. C07K 14/195; B33Y 10/00; B33Y 70/00; C07K 7/06; C07K 14/735; C12N 1/20; C12P 21/02; C12R 1/19

(52) U.S. Cl. CPC C07K 14/195; B33Y 10/00; B33Y 70/00; C07K 7/06; C07K 14/70535; C12N 1/20; C12P 21/02; C07K 2319/00; C12R 2001/19

(57) ABSTRACT

In one aspect, the disclosure relates to a platform for producing autogenic engineered living materials, the platform including at least a plurality of engineered cells, wherein each engineered cell expresses at least one β-solenoid protein monomer. In an aspect, the β-solenoid protein monomer can be a CsgA analog from a non-model organism such as, for example, an extremophile. In a further aspect, the β-solenoid protein monomer has customizable properties and can further be engineered to perform additional functions such as, for example, binding to specific target molecules. Also disclosed herein are a method for producing β-solenoid protein monomers by culturing the disclosed platform, hydrogels incorporating the β-solenoid monomers, and methods for 3D printing using the disclosed hydrogels as inks.

### CROSS-REFERENCE TO RELATED APPLICATIONS

This application claims the benefit of U.S. Provisional Application Ser. No. 63/680,451, filed Aug. 7, 2024, which is incorporated herein by reference in its entirety.

### CROSS REFERENCE TO SEQUENCE LISTING

The genetic components described herein are referred to by sequence identifier numbers (SEQ ID NO). The sequence listing in xml format is provided as a file named “222207-1270_Sequence_Listing.xml” created on Aug. 4, 2025, and having a size of 37,470 bytes, is incorporated by reference in its entirety.

### BACKGROUND

Engineered living materials (ELMs) is a rapidly growing field wherein living cells are engineered to produce materials with life-like functionalities for sustainability, biomedicine, biosensing, biomining, and bioremediation applications. Based on the polymeric matrix, ELMs can be broadly classified into two types, namely, exogenic and autogenic ELMs. In exogenic ELMs, living cells are embedded in a synthetic/natural polymeric matrix obtained from an external source, whereas, in autogenic ELMs, the living cells are utilized/engineered for in situ production of native/functional polymeric matrix. Evidently, autogenic ELMs are advantageous over exogenic ELMs, as they enable on-demand in situ biosynthesis, self-assembly, self-organization, self-regeneration, self-regulation, environmental adaptability, and functionalization of the polymeric matrix. Although the autogenic ELMs are crucial to realize the full potential of this emerging technology, there are only a few reported autogenic ELMs, emphasizing the need for innovative design strategies to 1) harness the programmability and biomanufacturing capabilities of living cells and 2) discover the wide variety of functionalities prevalent in the natural systems.

In the existing autogenic ELMs, the native extracellular matrix (ECM) of bacterial biofilms is utilized either directly or after functionalization. For example, the native protein nanofibers-based ECM of Escherichia coli and Bacillus subtilis, which include CsgA and TasA, respectively, have been genetically modified by fusing them with the desired protein domains to obtain functional autogenic ELMs. Alternatively, the native cellulose nanofibers-based ECM of Komagataeibacter rhaeticus has been employed to obtain autogenic ELMs, but unlike the above example of protein-based ECM, the genetic functionalization of cellulose/polysaccharide to controllably modulate its properties is still elusive. Furthermore, the genetic modification of surface-layer proteins of E. coli, B. subtilis, and Caulobacter crescentus has resulted in autogenic ELMs, but they have limited scope to tailor physical, chemical, and biological properties in comparison to functional nanofiber-based ECM.

Despite advances in ELM research, there is still a scarcity of systems and methods for producing ELMs that can be tailored to achieve a broad range of physical, chemical, and/or biological properties. These needs and other needs are satisfied by the present disclosure.

### SUMMARY

In accordance with the purpose(s) of the present disclosure, as embodied and broadly described herein, the disclosure, in one aspect, relates to a platform for producing autogenic engineered living materials, the platform including at least a plurality of engineered cells, wherein each engineered cell expresses at least one β-solenoid protein monomer. In an aspect, the β-solenoid protein monomer can be a CsgA analog from a non-model organism such as, for example, an extremophile. In a further aspect, the β-solenoid protein monomer has customizable properties and can further be engineered to perform additional functions such as, for example, binding to specific target molecules. Also disclosed herein are a method for producing β-solenoid protein monomers by culturing the disclosed platform, hydrogels incorporating the β-solenoid monomers, and methods for 3D printing using the disclosed hydrogels as inks.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic of the SECRETE platform to produce CsgA-like de novo β-solenoid proteins for autogenic engineered living functional materials. CsgA-like proteins discovered by mining the National Center for Biotechnology Information (NCBI) protein database were studied by using AlphaFold2 and molecular dynamics (MD) simulations to predict their structure. E. coli curli machinery was hacked to produce de novo β-solenoid proteins of non-model extremophilic bacteria to obtain materials with modular physico-chemical properties, programmable biofunctionalities, and tunable mechanical properties.

FIGS. 2A-2F show structures of β-solenoid protein building blocks predicted by AlphaFold2. The predicted β-solenoid protein models of (FIG. 2A) CsgA, (FIG. 2B) Hs13-CsgA, (FIG. 2C) Am18-CsgA, (FIG. 2D) Bc36-CsgA, (FIG. 2E) El43-CsgA, and (FIG. 2F) Er46-CsgA. The longitudinal view of β-solenoid protein model shows the number of cross-β repeats. Each β-solenoid protein sequence is presented to showcase the conserved (N, Q, G indicates >50% of conservation probability) and non-conserved residues (n, g indicates 25-50% of conservation probability and for <25%, it is indicated as X). On-axis top view shows the β-solenoid fold with inward-facing residues (green), key residues (blue), non-conserved residues (red), and outlier regions (orange). For clarity, only the top 5 cross-β repeats are shown for the on-axis top view.

FIGS. 3A-3G show physicochemical characteristics of β-solenoid proteins and their structure stability analysis by all-atom molecular dynamics simulations. Comparison of the physicochemical characteristics of β-solenoid proteins, (FIG. 3A) molecular weight, (FIG. 3B) isoelectric point, (FIG. 3C) net charge, and (FIG. 3D) hydrophobicity index. (FIG. 3E) Root mean square deviation (RMSD) of the β-strands for β-solenoid proteins throughout MD simulation. (FIG. 3F) Hydrogen bond autocorrelation function for β-solenoid proteins. (FIG. 3G) Average normalized van der Waals, electrostatic, and total interaction energies (IE). IE1: between KR and KR, IE2: KR and all residues, and IE3: non-KR and all residues, over the period of the production run. The energies were normalized per residue energies.

FIGS. 4A-4E show the SECRETE platform to produce β-solenoid proteins of non-model organisms by hacking the curli secretion machinery of E. coli. (FIG. 4A) Congo red assay to determine the β-solenoid proteins produced by engineered E. coli. Biological replicates n=4. Data represented as mean±standard deviation. **** p≤0.0001, one-way ANOVA followed by Dunnett's test. (FIG. 4B) Wide-angle X-ray scattering (WAXS) analysis revealed cross-β characteristics of β-solenoid proteins. (FIG. 4C) d-spacing (interplanar) values of 0.98 nm and 0.46 nm, corresponding to inter-β-sheet and intra-β-strand distances. (FIG. 4D) Field-emission scanning electron microscopy (FESEM) images show the β-solenoid protein nanofibers self-assembled in the extracellular milieu. Scale bar 1 μm. (FIG. 4E) Congo red birefringence showed the cross-β characteristics of β-solenoid protein nanofibers. Scale bar 50 μm.

FIGS. 5A-5E show biomanufacturing of macroscopic autogenic engineered living materials from various β-solenoid proteins. (FIG. 5A) Field-emission scanning electron microscopy (FESEM) images of β-solenoid protein hydrogels. Scale bar 1 μm. (FIG. 5B) Optical images of hydrogel and film manufactured using β-solenoid protein nanofibers. Scale bar 0.5 cm. (FIG. 5C) Yield of β-solenoid protein hydrogel obtained from engineered E. coli using filtration protocol. Biological replicates n=4. Data represented as mean±standard deviation. ****p≤0.0001, *** p≤0.0009 one-way ANOVA followed by Dunnett's test. (FIG. 5D) Storage modulus of β-solenoid protein hydrogel. Biological replicates n=3. Data represented as mean±standard deviation. *** p≤0.001, *p≤0.014 one-way ANOVA followed by Dunnett's test. (FIG. 5E) 3D printing of Am18-CsgA protein hydrogel. Scale bar 1 cm.

FIGS. 6A-6B show programming the biological functionalities of de novo β-solenoid proteins. (FIG. 6A) Genetic design of E. coli programmed to produce β-solenoid protein nanofibers displaying iron oxide binding peptide (Hs13-CsgA-IronBP) along with FESEM image of the protein nanofibers self-assembled in the extracellular milieu. Weight analysis and energy dispersive X-ray (EDAX) analysis show the enhanced binding of iron oxide nanoparticles to engineered nanofibers, Hs13-CsgA-IronBP. Scale bar 1 μm. Biological replicates n=4. Data represented as mean±standard deviation. *** p≤0.0001 two-way ANOVA for EDAX analysis and *** p≤0.0008 t-test followed by Welch's test for weight analysis. (FIG. 6B) Genetic design of E. coli cells to produce β-solenoid protein nanofibers displaying IgG antibody binding domain (Hs13-CsgA-IgG-BD) along with FESEM image of the protein nanofibers self-assembled in the extracellular milieu. Fluorescent IgG antibodies binding assay shows higher binding for Hs13-CsgA-IgG-BD than wildtype Hs13-CsgA nanofibers. Scale bar 1 μm. Biological replicates n=3. Data represented as mean±standard deviation. ** p≤0.0024 t-test followed by Welch's test. The structures of de novo β-solenoid proteins, Hs13-CsgA-IronBP and Hs13-CsgA-IgG-BD were predicted using AlphaFold2.

FIG. 7 shows genetically modified CsgA structure prediction using AlphaFold2. The protein structure of CsgA, with C-terminal fusion of Trefoil Factor 2 (TFF2) predicted by AlphaFold2, shows the β-solenoid fold of CsgA.

FIG. 8 shows AlphaFold2 structure prediction of β-solenoid proteins. Predicted structure of 50 random sequences obtained from protein mining. The prediction shows β-solenoid fold with cross-βrepeat units ranging from 5 to 46.

FIG. 9 shows top view of β-solenoid proteins. Interloop distances of the β-solenoid proteins.

FIG. 10 shows gravy hydrophobicity index of β-solenoid proteins. The hydrophobicity of CsgA, Hs13-CsgA, Am18-CsgA, Bc36-CsgA, El43-CsgA and Er46-CsgA is presented along with their Gravy index.

FIG. 11 shows the structure of β-solenoid proteins predicted by AlphaFold2. Structure of CsgA, Hs13-CsgA, Am18-CsgA, Bc36-CsgA, El43-CsgA and Er46-CsgA predicted by AlphaFold2 along with their pLDDT (predicted local distance difference test) scores.

FIG. 12 shows a comparison of β-solenoid protein structures obtained using AlphaFold2 and molecular dynamics simulation. The structures of CsgA, Hs13-CsgA, Am18-CsgA, Bc36-CsgA, El43-CsgA, and Er46-CsgA predicted using AlphaFold2 and molecular dynamics simulation are overlaid to show their similarity. The corresponding pLDDT (predicted local distance difference test) scores for the entire β-solenoid protein and RMSD (root mean square deviation) values of the β-solenoid core (without outlier regions and N-terminal) are presented.

FIG. 13 shows RMSD of the β-solenoid proteins. Molecular dynamics simulation based RMSD (root mean square deviation) values for 150 ns of entire CsgA, Hs13-CsgA, Am18-CsgA, Bc36-CsgA, El43-CsgA and Er46-CsgA.

FIG. 14 shows RMSF of the β-solenoid proteins. Molecular dynamics simulation based RMSF (root mean squared fluctuations) values of entire CsgA, Hs13-CsgA, Am18-CsgA, Bc36-CsgA, El43-CsgA and Er46-CsgA. The dark lines show the average RMSF over the last 50 ns production run and the standard deviations are shown in lighter shades.

FIG. 15 shows Ramachandran plots of β-solenoid proteins. The Ramachandran plots show the dihedral angles ϕ and ψ for CsgA, Hs13-CsgA, Am18-CsgA, Bc36-CsgA, El43-CsgA and Er46-CsgA, as a function of simulation time intervals.

FIG. 16 shows amino acid composition of β-solenoid proteins. The plots show the percentage of each amino acid present in CsgA, Hs13-CsgA, Am18-CsgA, Bc36-CsgA, El43-CsgA and Er46-CsgA. The positively charged residues (R and K) and the negatively charged residues (D and E) are highlighted in blue and red, respectively. The inset of each plot lists the total number of amino acid residues (N), net charge (Q), and the charge per residue (Q/N).

FIGS. 17A-17B show 3D printing of β-solenoid protein hydrogels. The hydrogels of (FIG. 17A) Hs13-CsgA and (FIG. 17B) Er46-CsgA were utilized to show their utility for 3D printing. Scale bar 1 cm.

FIG. 18 shows AlphaFold2 structure prediction of engineered β-solenoid proteins. The structures of β-solenoid protein, Hs13-CsgA, that is genetically grafted with iron (Hs13-CsgA-IronBP) and antibody (Hs13-CsgA-IgG-BD) binding domains, predicted using AlphaFold2, is shown along with their pLDDT (predicted local distance difference test) scores.

FIG. 19 shows results of a Congo Red Assay. The plot shows the binding of Congo red dye to the engineered β-solenoid variants of Hs13-CsgA-IronBP and Hs13-CsgA-IgG-BD, indicating their cross-β characteristics. Biological replicates n=4. Data represented as mean±standard deviation. **** p≤0.0001, one-way ANOVA followed by Dunnett's test.

FIG. 20 shows energy dispersive X-ray analysis (EDAX) for elemental composition. Representative EDAX plots of (FIG. 20A) Hs13-CsgA and (FIG. 20B) Hs13-CsgA-IronBP incubated with iron oxide nanoparticles show the elemental composition. Biological replicates n=4.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

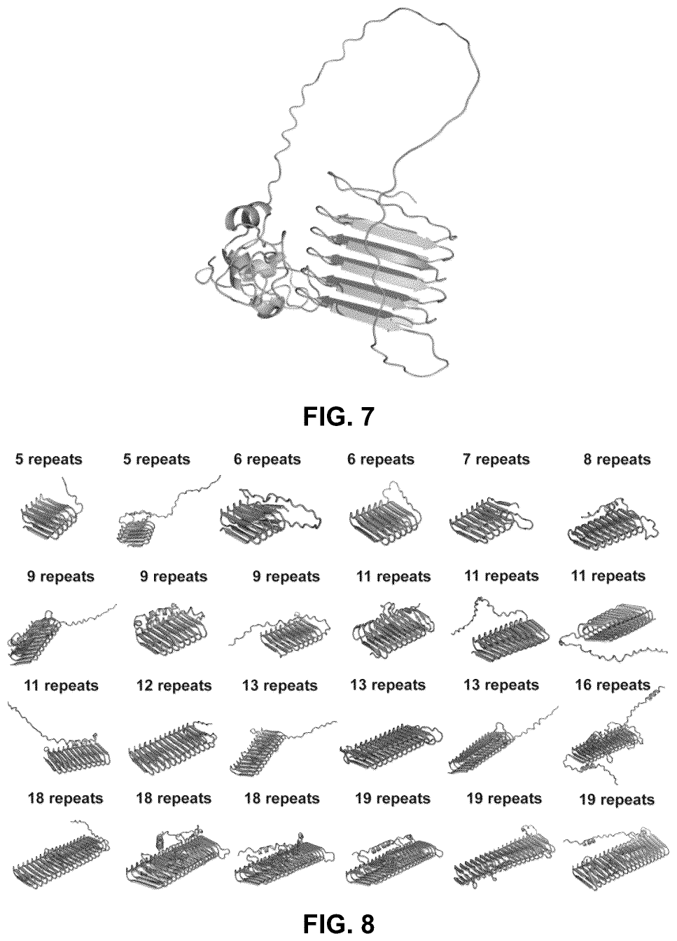

In the last twelve years, the ECM protein nanofibers of E. coli self-assembled from CsgA are the most studied autogenic ELMs. These protein nanofibers, commonly known as curli, are attractive due to their 1) resistance to heat, solvents, pH, detergents, and denaturants, 2) mechanical properties, and 3) ability to modulate functionalities by genetically grafting the desired protein domains. CsgA, with its cross-β structure, is energetically among the most stable protein folds, while the β-solenoid architecture facilitates the positioning of the C- and N-terminal on its periphery. As a result, the peptide/protein domains genetically grafted to CsgA via a flexible linker are often found to fold independently to their functional form (FIG. 7). Moreover, the head-to-tail stacking of CsgA building blocks leads to the formation of curli nanofibers, and the genetically grafted peptide/protein domain gets displayed on its periphery to form the functional curli nanofibers, which have been demonstrated for various applications in the last several years. Thus, it was hypothesized that the β-solenoid architecture of the CsgA protein building block is crucial for the fascinating properties and applications of curli nanofibers-based autogenic ELMs.

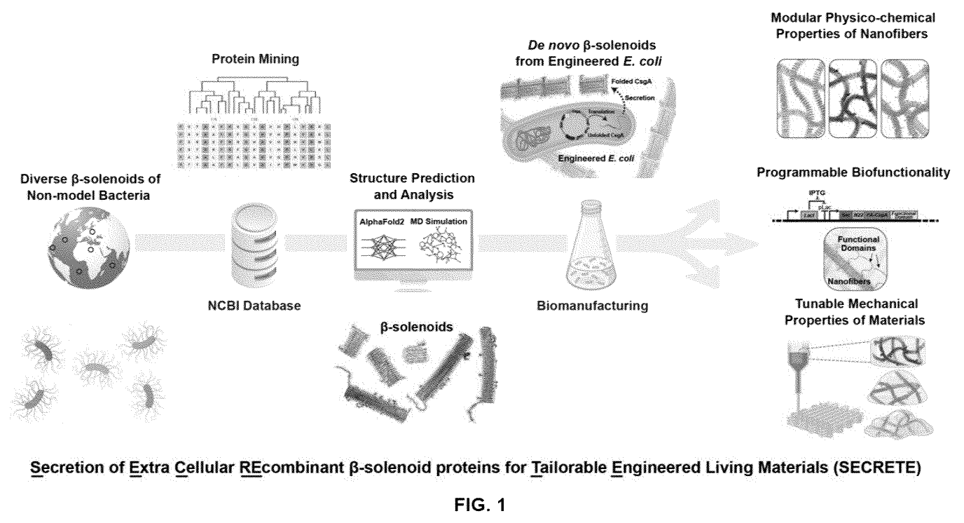

Herein are disclosed de novo autogenic functional ELMs, wherein the E. coli is engineered to produce de novo ECM of protein nanofibers (FIG. 1). In one aspect, protein database mining was conducted to discover CsgA-like β-solenoid proteins that can serve as de novo building blocks of synthetic ECM. In a further aspect, the artificial intelligence (AI) tool AlphaFold2 was used to predict the structure of these de novo β-solenoid proteins, and the stability of the folded structure was deciphered using all-atom (AA) molecular dynamics (MD) simulations in the explicit water model. In one aspect, since these β-solenoid proteins were from non-model organisms thriving in different parts of the globe, the curli secretion machinery of model organism E. coli was modified to produce de novo ECM. In a further aspect, these ECMs were employed to produce functional materials like hydrogels/films and show that their physicochemical properties can be tailored. In another aspect, herein it is demonstrated that the biological functions of these de novo ECM-based ELMs could be controllably modified by genetically grafting the desired protein domains to facilitate specific binding to nanoparticles or antibodies.

### Platform for Autogenic Engineered Living Materials

In one aspect, disclosed herein is a platform for producing autogenic engineered living materials (ELM), the platform including at least a plurality of engineered cells, wherein each engineered cell of the plurality expresses at least one β-solenoid protein monomer. In a further aspect, the engineered cells have been engineered to express at least one β-solenoid protein monomer that is not native to the cells. In another aspect, the entire plurality of cells can express the same β-solenoid protein monomer. However, in an alternative aspect, the plurality of cells can include two or more sub-populations of cells, such as, for example, two, three, four, five, or more sub-populations of engineered cells, wherein each of the sub-populations expresses a different β-solenoid protein monomer.

In some aspects, the engineered cells can be Bacillus subtilis, Saccharomyces cerevisiae, Pichia pastoris, Pseudomonas putida, or E. coli. In a further aspect, E. coli strain PQN4 may be particularly useful, although other strains are contemplated and should be considered disclosed. In an aspect, the engineered cell can be modified to remove a native curli operon, thus allowing overexpression of the inserted β-solenoid protein monitor. In one aspect, the engineered cell contains a plasmid encoding the β-solenoid protein monomer. In a further aspect, the plasmid can be pET21d, although other plasmids are also contemplated and should be considered disclosed.

In any of these aspects, the β-solenoid protein monomer can be a CsgA analog from a non-model organism such as, for example, Halomonas saliphila, Alteromonas macleodii, Blastomonas sp. CACIA14H2, Erythrobacter longus, Ensifer sp. Root31, or any combination thereof. In one aspect, the non-model organism can be an extremophile or an organism that is difficult to culture and proliferate under laboratory conditions.

In some aspects, the β-solenoid protein monomer is engineered to include one or more native E. coli sequences to facilitate extracellular secretion, self-assembly, or both. In a further aspect, the one or more native E. coli sequences can be an N-terminal signal sequence (Sec; SEQ ID NO. 17), an N-terminal curli-specific targeting sequence (N22; SEQ ID NO. 18), or both Sec and N22.

### Cross-β Units

In any of these aspects, in the disclosed platform, the β-solenoid protein monomer includes a plurality of cross-β repeat units, wherein each cross-β repeat unit itself includes:
a first β-strand having a sequence KR₁-Ω-Ψ-Ω-Ψ-Ω-KR₇;
an intra-repeat loop having a sequence X-G-X-G,
a second β-strand having a sequence KR₁-Ω-Ψ-Ω-Ψ-Ω-KR₇; and
an inter-repeat loop having a sequence X-X-X-X;
   wherein X represents a variable amino acid,
   wherein KR₁ is selected from serine and asparagine;
   wherein KR₇ is glutamine;
   wherein Ω represents an outward-facing hydrophilic amino acid; and
   wherein Ψ represents an inward-facing hydrophobic amino acid.

Exemplary β-solenoid protein monomers having cross-beta repeat units as described herein are provided in the Sequence Listing. In an aspect, other β-solenoid protein monomers are also contemplated and should be considered disclosed. In one aspect, other β-solenoid protein monomers can be discovered in a database such as, for example, GenBank or GenPept, where homology searches of CsgA or another β-solenoid protein monomer can be conducted. In a further aspect, following conduction of a homology search, protein folding modeling tools can be used to analyze the structure of identified sequences as described herein in the Examples.

In one aspect, each Y can independently be selected from alanine, isoleucine, valine, leucine, phenylalanine, serine, and threonine. In a further aspect, the β-solenoid protein monomer comprises from about 10 to about 50 cross-β repeat units, or about 10, 15, 20, 25, 30, 35, 40, 45, or about 50 cross-β repeats, or a combination of any of the foregoing values, or a range encompassing any of the foregoing values. In one aspect, the number of cross-β repeats is 13, 18, 36, 43, or 46. In another the β-solenoid protein monomer has an isoelectric point of from about 3.3 to about 4.5, or of about 3.3, 3.4, 3.5, 3.6, 3.7, 3.8, 3.9, 4.0, 4.1, 4.2, 4.3, 4.4, or about 4.5, or a combination of any of the foregoing values, or a range encompassing any of the foregoing values. Other properties including hydrophobicity, charge, and the like are disclosed in the Examples.

### Functionalized β-Solenoid Protein Monomers

In some aspects, in the disclosed platform, the β-solenoid protein monomer can be engineered to contain at least one additional functional moiety. In one aspect, when multiple different β-solenoid protein monomers are expressed in the same plurality of engineered cells, some of them can be functionalized and others may not be, or different cells can express differently functionalized monomers. In an alternative aspect, all engineered cells can express the same β-solenoid protein monomer.

In one aspect, the at least one additional functional moiety can be an iron binding peptide. In another aspect, the at least one additional functional moiety can be an immunoglobulin G (IgG) binding protein. However, other functional moieties are contemplated and should be considered disclosed. In one aspect, proteins or peptides having a desired functionality can be identified in a database such as GenBank or GenPept and can be fused to the β-solenoid protein monomer using standard molecular biology techniques. In any of these aspects, the at least one additional functional moiety can be connected to the β-solenoid protein monomer via a flexible amino acid linker. In one aspect, the at least one additional functional moiety can be incorporated at an N-terminus of the β-solenoid protein monomer, at a C-terminus of the β-solenoid protein monomer, or internally within the β-solenoid protein monomer

### β-Solenoid Protein Monomers and Method for Producing 3-Solenoid Protein Monomers

In one aspect, disclosed herein is a method for producing β-solenoid protein monomers, the method comprising culturing the disclosed platform. In a further aspect, culturing the platform includes the step of incubating the engineered cells in an appropriate culture medium for at least about 48 h. In another aspect, however, other culture times are also contemplated and should be considered disclosed based on research and production needs. For example, in one aspect, if a lower amount of β-solenoid protein monomer is needed, a shorter culture time is contemplated, whereas times longer than 48 hours may be used when a larger amount of β-solenoid protein monomer is desired. In one aspect, the culture medium is Luria-Bertani (LB) broth, although other nutrient solutions and culture media are also contemplated and should be considered disclosed. In a further aspect, other nutrients may be supplemented or eliminated from the culture medium.

Also disclosed herein is a plurality of β-solenoid protein monomers produced by the disclosed method. In one aspect, the β-solenoid protein monomers have at least about 95% sequence identity to any one of SEQ ID NOs. 3, 5, 7, or 9, or at least 99% sequence identity or 99.9% sequence identity. In a further aspect, variation in sequence identity may be found in loops and non-β-solenoid regions, or at the ends of the proteins as introduced by standard molecular biology techniques.

In one aspect, each individual β-solenoid protein monomer of the plurality can include at least one additional functional moiety. In a further aspect, the at least one additional functional moiety can be an iron-binding peptide and the β-solenoid protein monitor has at least about 95% sequence identity to SEQ ID NO. 11. In another aspect, the at least one additional functional moiety can be an IgG antibody binding protein and the β-solenoid protein monomer has at least about 95% sequence identity with SEQ ID NO. 13.

### Applications of the β-Solenoid Protein Monomers

In one aspect, disclosed herein is an amyloid nanofiber made from a plurality of β-solenoid protein monomers as disclosed herein.

In another aspect, disclosed herein is a hydrogel including disclosed β-solenoid protein monomers and/or disclosed amyloid nanofibers. In one aspect, the hydrogel exhibits shear-thinning behavior. Also disclosed herein are aquaplastics including the disclosed hydrogels. In one aspect, methods for making the hydrogels and aquaplastics are provided in US Patent Ser. No. 11,098,133.

In one aspect, disclosed herein is a method for 3D printing, the method including at least the step of extruding a disclosed hydrogel through a nozzle using a layer-by-layer approach to build a three-dimensional article. In another aspect, disclosed herein is a three-dimensional article produced by the disclosed method.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cross-β repeat unit,” “a non-model organism,” or “an amyloid nanofiber,” include, but are not limited to, mixtures, combinations, or series of two or more such cross-β repeat units, non-model organisms, or amyloid nanofibers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

A “β-solenoid protein monomer” as used herein refers to a protein having repeating beta strands, typically arranged in an antiparallel fashion to form a superhelix. In an aspect, in β-solenoid protein monomers, the beta strands form beta sheets. β-solenoid protein monomers can be identified in databases by searching for specific repeating motifs as described elsewhere herein, and are commonly found in a variety of prokaryotic organisms. β-solenoid protein monomers can assemble into larger structures including amyloid nanofibers, macroscopic structures, and the like, and can be genetically fused to other functional moieties to produce nanofibers with specific properties (e.g. iron binding).

A “non-model organism” as used herein refers to an organism, such as a prokaryotic species, that cannot normally be cultured or proliferated under standard laboratory conditions. In some aspects, non-model organisms can be extremophiles such as thermophiles, halophiles, or the like. In an aspect, peptides or proteins from non-model organisms can be engineered into model organisms such as, for example, E. coli, for the purpose of mass production of the peptides or proteins.

An “amyloid nanofiber” as used herein refers to fibrous protein aggregates. Formation of amyloid nanofiber structures occurs through noncovalent interactions, namely extended β-sheet hydrogen bond networks. Monomers such as β-solenoid protein monomers can self-assemble and pack together to form nanofibers. In one aspect, an amyloid nanofiber is straight and unbranched, although the disclosed amyloid nanofibers can be made from functionalized β-solenoid protein monomers in order to take on nonstandard structural features.

As used herein, “engineered living materials” or “ELMs” refers to the use of living cells to produce materials with life-like functionalities. “Autogenic” ELMs use living cells to produce a functional polymeric matrix. In some aspects, the functional polymeric matrix produced by ELMs can mimic the native extracellular matrix (ECM) of bacterial biofilms and may self-assemble, self-organize, self-heal, and self-regulate, in addition to being environmentally adaptable. In some cases, microorganisms producing the ELMs can be engineered to more efficiently produce the ELMs, or can include genes expressing fusion proteins containing polymeric components with added functionalities.

A “functional moiety” refers to a peptide or protein sequence having a specific function as used herein. In one aspect, the functional moiety is fused to the β-solenoid protein monomer. In a further aspect, the functional moiety can be fused at the N or C terminus of the β-solenoid protein monomer or can be inserted in to the core of the β-solenoid sequence. In another aspect, the functional moiety can add one or more functions to one β-solenoid protein monomer to produce ELMs with different properties such as, for example, heavy-metal binding or sequestration, including of specific metals, antibody binding, and the like.

As used herein, a “hydrogel” refers to a network made from a hydrophobic polymer physically crosslinked by a water-soluble polymer. In an aspect, the water-soluble portions of the hydrogel can retain water or other fluids (e.g. cell culture medium) and may be flexible and/or have swelling properties. In some aspects, hydrogels can exhibit shear-thinning behavior and may be extruded, 3D printed, or the like.

As used herein, “shear-thinning behavior” refers to a property of a material such as a hydrogel, wherein the material behaves like a fluid under external stress (i.e., exhibits a lower viscosity, enabling flow) and reverts to an initial, higher viscosity when the external stress is removed. In an aspect, a hydrogel exhibiting shear-thinning behavior can be extruded through a small opening such as found in a 3D print head or nozzle.

An “aquaplastic” is a biodegradable bioplastic material. Aquaplastics are based on microbial biofilms and can be processed in water. In one aspect, aquaplastics are environmentally benign and can be used as a replacement for petroleum-based plastics in many instances.

A “layer-by-layer” approach is a technique for synthesizing materials by 3D printing or another means. In a typical layer-by-layer approach, a first layer of film is deposited and, if necessary crosslinked. Subsequent layers are added in the same way until a 3D article has been constructed. Wash steps may or may not be performed between deposition of individual layers. Layer-by-layer approaches may be particularly useful for irregularly-shaped objects; each layer may have a slightly different shape or surface area, but finds support on the layer(s) below.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A platform for producing autogenic engineered living materials (ELM), the platform comprising a plurality of engineered cells, wherein each engineered cell of the plurality expresses at least one non-native β-solenoid protein monomer, wherein the at least one β-solenoid protein monomer comprises a CsgA analog from a non model organism.

Aspect 2. The platform of aspect 1, wherein the at least one β-solenoid protein monomer expressed by each engineered cell of the plurality is the same.

Aspect 3. The platform of aspect 1, wherein the plurality of engineered cells comprises two or more sub-populations of engineered cells, wherein each of the two or more sub-populations expresses a different β-solenoid protein monomer.

Aspect 4. The platform of any one of aspects 1-3, wherein the engineered cell comprises Bacillus subtilis, Saccharomyces cerevisiae, Pichia pastoris, Pseudomonas putida, or E. coli.

Aspect 5. The platform of aspect 4, wherein the E. coli comprises strain nissle or PQN4.

Aspect 6. The platform of any one of aspects 1-5, wherein the engineered cell has been modified to remove a native curli operon.

Aspect 7. The platform of any one of aspects 1-6, wherein the engineered cell comprises a plasmid encoding at least the β-solenoid protein monomer.

Aspect 8. The platform of aspect 7, wherein the plasmid comprises pET21d.

Aspect 9. The platform of any one of aspects 1-8, wherein the non-model organism comprises Halomonas saliphila, Alteromonas macleodii, Blastomonas sp. CACIA14H2, Erythrobacter longus, Ensifer sp. Root31, or any combination thereof.

Aspect 10. The platform of any one of aspects 1-9, wherein the β-solenoid protein monomer is engineered to include one or more native E. coli sequences to facilitate extracellular secretion, self-assembly, or both.

Aspect 11. The platform of aspect 10, wherein the one or more native E. coli sequences comprise an N-terminal signal sequence (Sec), an N-terminal curli-specific targeting sequence (N22), or both Sec and N22.

Aspect 12. The platform of any one of aspects 1-11, wherein the β-solenoid protein monomer comprises a plurality of cross-β repeat units, wherein each cross-β repeat unit comprises:
a first β-strand having a sequence KR₁-Ω-Ψ-Ω-Ψ-Ω-KR₇;
an intra-repeat loop having a sequence X-G-X-G,
a second β-strand having a sequence KR₁-Ω-Ψ-Ω-Ψ-Ω-KR₇; and
an inter-repeat loop having a sequence X-X-X-X;
   wherein X represents a variable amino acid,
   wherein KR₁ is selected from serine and asparagine;
   wherein KR₇ is glutamine;
   wherein Ω represents an outward-facing hydrophilic amino acid; and
   wherein Ψ represents an inward-facing hydrophobic amino acid.

Aspect 13. The platform of aspect 12, wherein each Y is independently selected from alanine, isoleucine, valine, leucine, phenylalanine, serine, and threonine.

Aspect 14. The platform of aspect 12 or 13, wherein the β-solenoid protein monomer comprises from about 10 to about 50 cross-β repeat units.

Aspect 15. The platform of any one of aspects 1-14, wherein the β-solenoid protein monomer has an isoelectric point of from about 3.3 to about 4.5.

Aspect 16. The platform of any one of aspects 1-15, wherein the β-solenoid protein monomer is engineered to contain at least one additional functional moiety.

Aspect 17. The platform of aspect 16, wherein the at least one additional functional moiety is incorporated at an N-terminus of the β-solenoid protein monomer, at a C-terminus of the β-solenoid protein monomer, or internally within the β-solenoid protein monomer.

Aspect 18. The platform of aspect 16 or 17, wherein the at least one additional functional moiety comprises an iron binding peptide.

Aspect 19. The platform of aspect 16 or 17, wherein the at least one additional functional moiety comprises an immunoglobulin G (IgG) binding protein.

Aspect 20. The platform of any one of aspects 16-19, wherein the at least one additional functional moiety is connected to the β-solenoid protein monomer via a flexible amino acid linker.

Aspect 21. A method for producing β-solenoid protein monomers, the method comprising culturing the platform of any one of aspects 1-20.

Aspect 22. The method of aspect 21, wherein culturing the platform comprises incubating the engineered cells in an appropriate culture medium for at least about 48 h.

Aspect 23. A plurality of β-solenoid protein monomers produced by the method of aspect 21 or 22.

Aspect 24. The plurality of β-solenoid protein monomers of aspect 23, wherein the β-solenoid protein monomers have at least about 95% sequence identity to any one of SEQ ID NOs. 3, 5, 7, or 9.

Aspect 25. The plurality of β-solenoid protein monomers of aspect 23, wherein each individual β-solenoid protein monomer of the plurality comprises at least one additional functional moiety.

Aspect 26. The plurality of β-solenoid protein monomers of aspect 25, wherein the at least one additional functional moiety comprises an iron-binding peptide.

Aspect 27. The plurality of β-solenoid protein monomers of aspect 26, wherein the β-solenoid protein monomers have at least about 95% sequence identity to SEQ ID NO. 11.

Aspect 28. The plurality of β-solenoid protein monomers of aspect 23, wherein the at least one additional functional moiety comprises an IgG antibody binding protein.

Aspect 29. The plurality of β-solenoid protein monomers of aspect 28, wherein the β-solenoid protein monomers have at least about 95% sequence identity with SEQ ID NO. 13.

Aspect 30. An amyloid nanofiber comprising the plurality of β-solenoid protein monomers of any one of aspects 23-29.

Aspect 31. A hydrogel comprising the plurality of β-solenoid protein monomers of aspect 23-29 or the amyloid nanofiber of aspect 30.

Aspect 32. The hydrogel of aspect 31, wherein the hydrogel exhibits shear-thinning behavior.

Aspect 33. An aquaplastic comprising the hydrogel of aspect 32.

Aspect 34. A method for 3D printing, the method comprising extruding the hydrogel of aspect 31 or 32 through a nozzle using a layer-by-layer approach to build a three-dimensional article.

Aspect 35. A three-dimensional article produced by the method of aspect 34.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Results

### Protein Mining and Structure Prediction

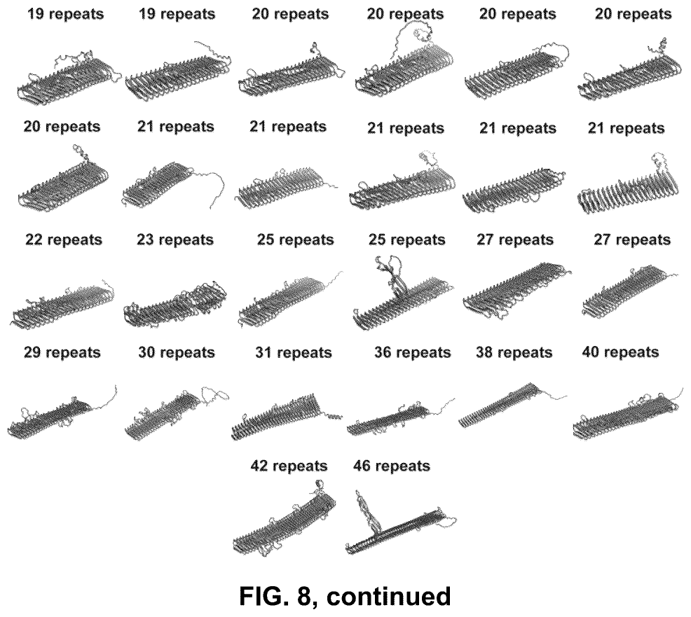

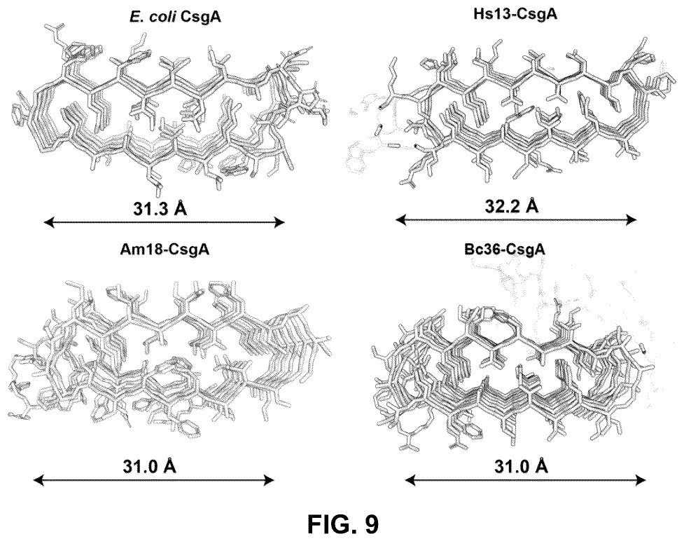

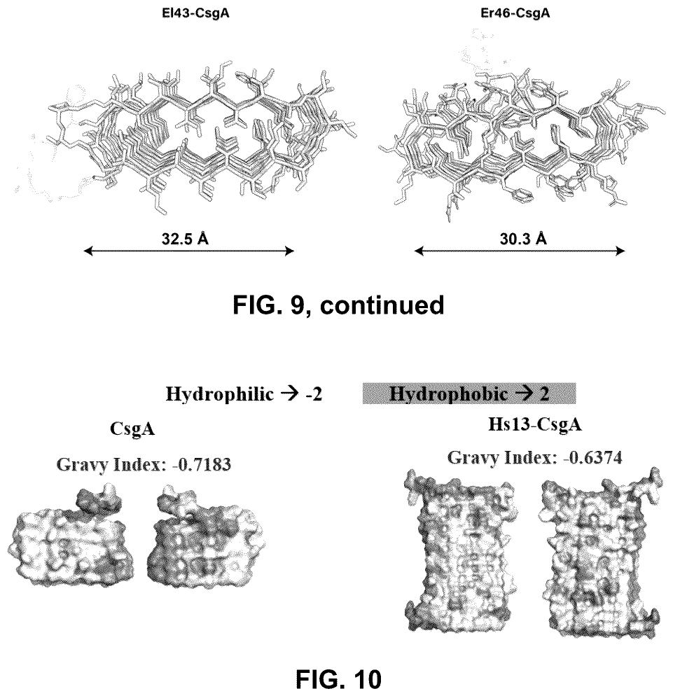

CsgA is a 13 kDa protein of 131 amino acids with five cross-β repeats, wherein the minimalistic curli repeat sequence is presented in the form X₆QXGX₂NX₁₀. To identify CsgA-like proteins, the National Center for Biotechnology Information (NCBI) database was used to search for proteins having X₆QXGX₂NX₁₀ sequence, which resulted in 33,564 entries and their lengths varied from 141 to 1390 amino acids. From these, 50 protein sequences were randomly selected, and their protein structure was predicted using AlphaFold2, which revealed β-solenoid architecture for all of them (FIG. 8). Finally, for a detailed study, five protein sequences of different lengths that belonged to diverse bacteria species were selected, namely, Halomonas saliphila (Hs13-CsgA), Alteromonas macleodii (Am18-CsgA), Blastomonas sp. CACIA14H2 (Bc36-CsgA), Erythrobacter longus (El43-CsgA), and Ensifer sp. Root31 (Er46-CsgA). These CsgA homologs, named according to their genus, species, and predicted number of cross-β repeats, represent a range of ecological adaptations and functional properties, none of which had been experimentally validated for curli production prior to this study.

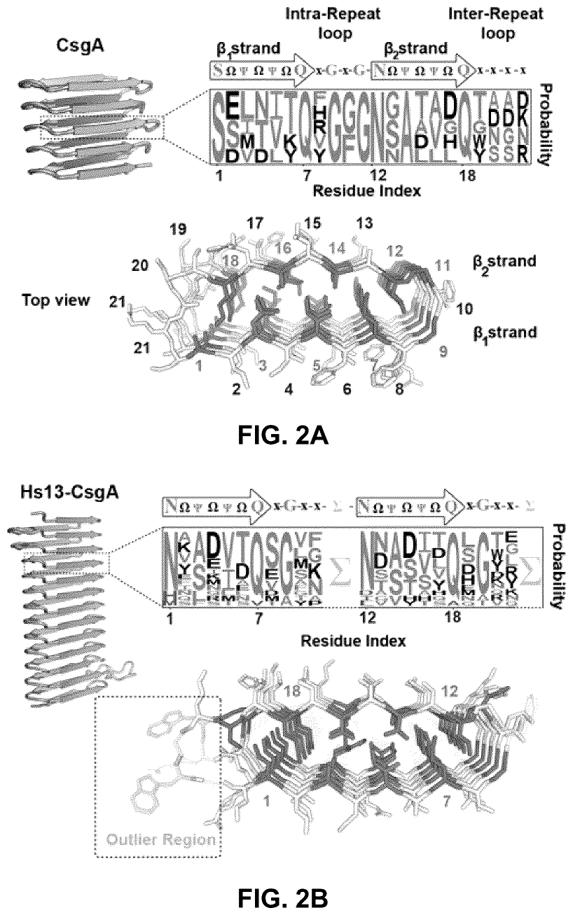

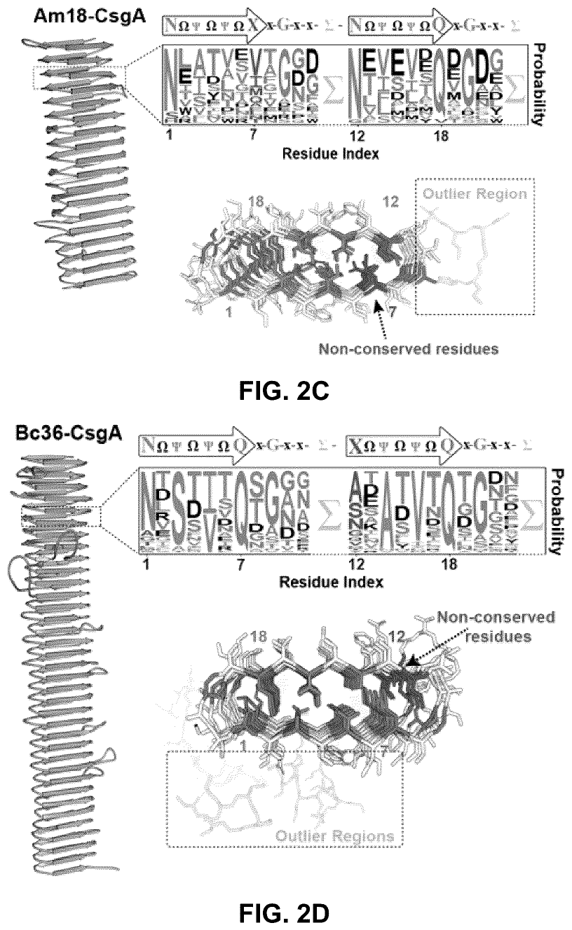

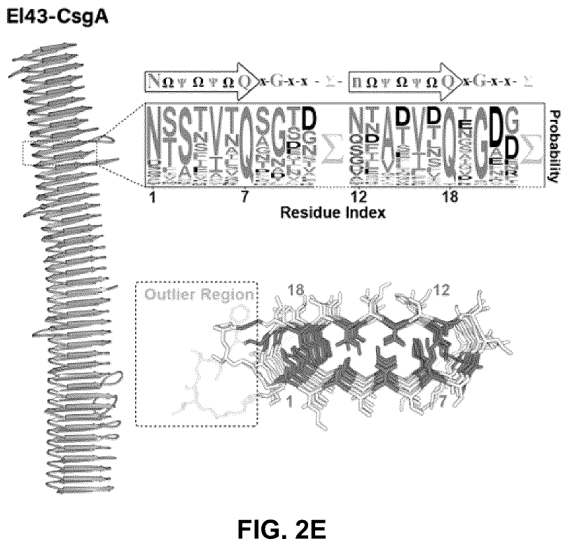

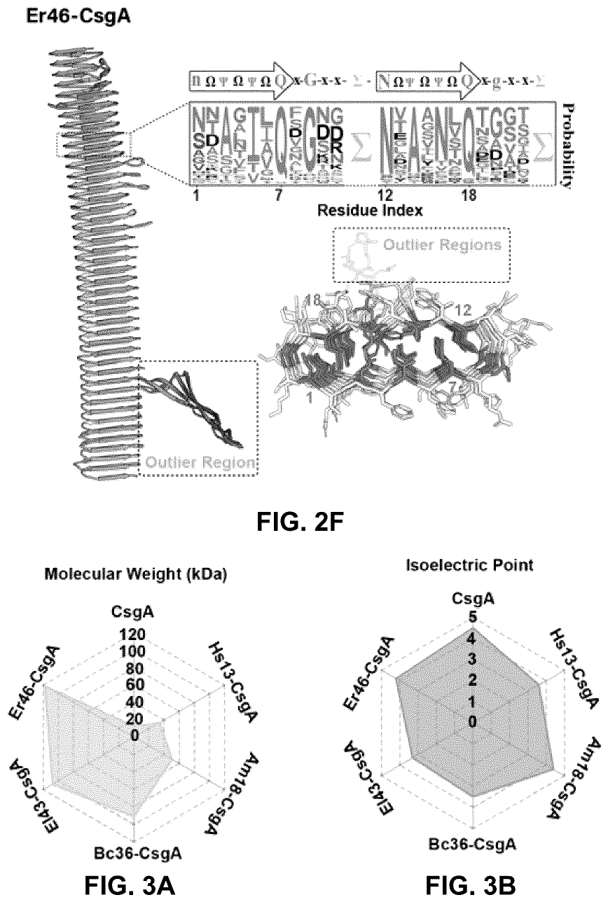

The 3D structure of CsgA and five other CsgA homologs predicted using AlphaFold2 revealed the signature sequence KR₁-Ω-Ψ-Ω-Ψ-Ω-KR₇ of cross-β sheet structure, wherein Ω represents outward-facing residues, while KR₁, KR₇, and Ψ represent inward-facing residues (FIGS. 2A-F and 8). Each repeat unit in CsgA consists of two antiparallel β-strands, β₁, and β₂, arranged in a cross-β sheet structure, with each β-strand made of seven residues from KR₁ to KR₇ that align across the entire folded monomer. These β-strands within each repeat unit are connected by short loops. The first loop, connecting β₁ to β₂ within a repeat unit, is referred to as the intra-repeat loop, while the loop connecting one repeat unit to the next is called the inter-repeat loop. Both loops are typically composed of four residues, while the intra-repeat loop is represented by the sequence X-G-X-G, where X represents variable residues, and G signifies the conserved glycine, which allows for structural flexibility. In summary, the conserved β-strand and loop sequence of each repeat unit follows the pattern: β₁ strand (KR₁-Ω-Ψ-Ω-Ψ-Ω-KR₇), intra-repeat loop (X-G-X-G), β₂ strand (KR₁-Ω-Ψ-Ω-Ψ-Ω-KR₇), inter-repeat loop (X-X-X-X), and so on. Different CsgA homologs vary in the number of repeat units: E. coli CsgA consists of 5 repeat units, while Hs13-CsgA has 13, Am18-CsgA has 18, Bc36-CsgA has 36, El43-CsgA has 43, and Er46-CsgA has 46 repeat units (43 in the core β-solenoid structure and 3 in the outlier region).

Within each β-strand pair (β₁ and β₂) in a repeat unit, key residues (KR), located at the beginning (KR₁) and the end (KR₇) of each β-strand, are reported to play a crucial role in stabilizing and ensuring proper folding of the monomer. In E. coli CsgA, KR₁ of β₁ is conserved as serine (S), while KR₁ of 32 is asparagine (N), and KR₇ of both β₁ and β₂ is glutamine (Q). However, these key residues KR₁/KR₇ in β₁/β₂ strands are conserved to various extents for the other five β-solenoids, and the non-conserved key residues, denoted as X in the consensus motif, are highlighted in red. The inward-facing residues, Ψ, shown in green, are tightly packed between the β-sheets, stabilizing the fibril core. These inward-facing residues typically consist of hydrophobic amino acids (A, I, V, L, F), as well as serine (S) or threonine (T). Meanwhile, outward-facing residues, Q, shown in black, are usually hydrophilic and exposed to the surrounding environment, facilitating fibrillation. Additionally, the structural analysis revealed outlier regions (highlighted in orange and denoted as Σ), which extend beyond the canonical β-strands and loop motifs, are observed in all five variants, except for E. coli CsgA. While the precise role of these outlier regions remains unclear, they may have functional implications or impact the overall architecture of the fibrils, warranting further investigation.

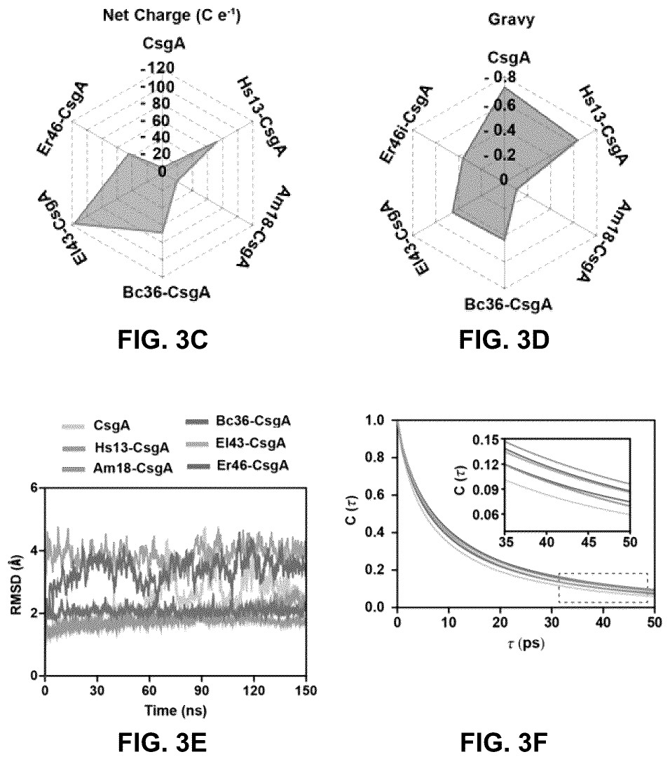

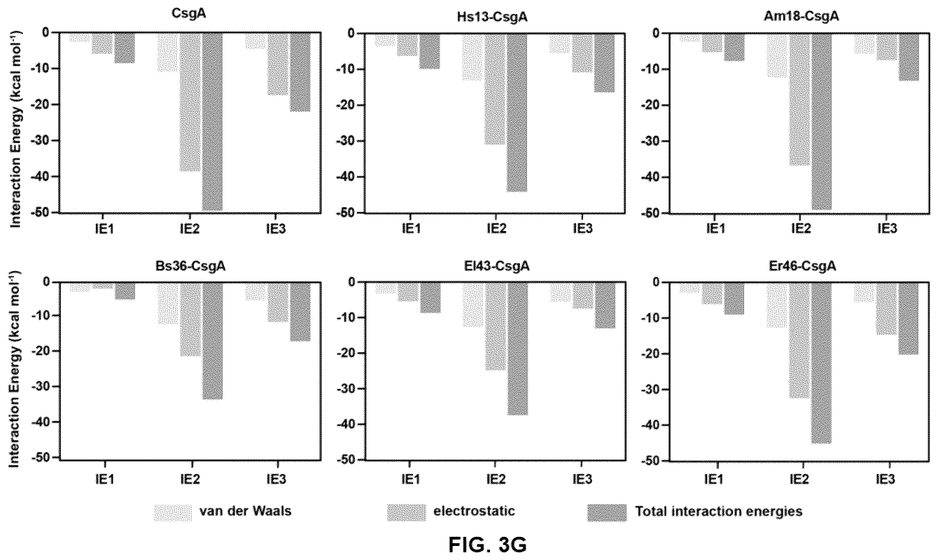

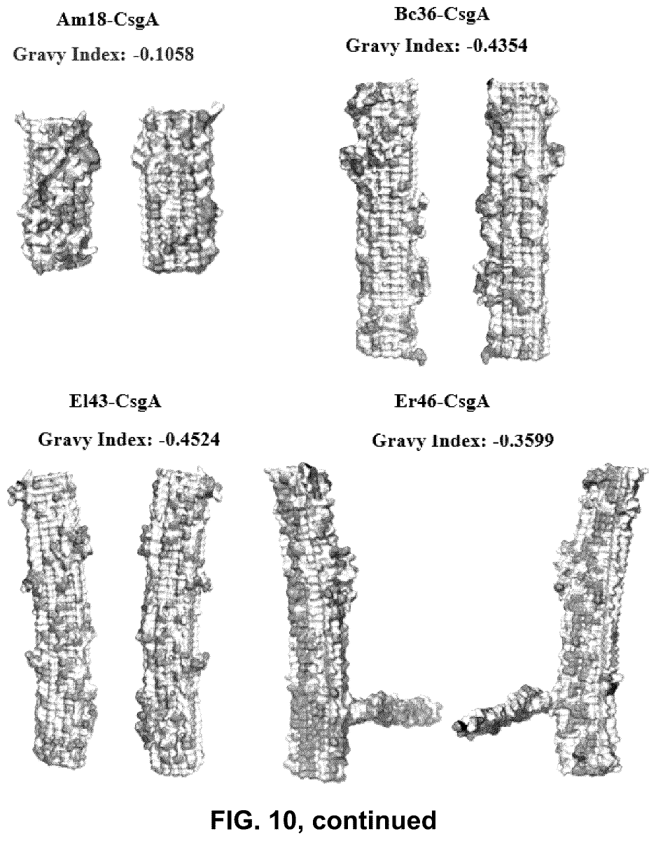

Additional analysis shed insights into the physicochemical characteristics of these six β-solenoid proteins (FIGS. 3A-3D, Tables 1-2). The molecular weight of the proteins increased with a greater number of repeats, from 13.1 kDa for CsgA to 117.6 kDa for Er46-CsgA, while their isoelectric points remained similar, ranging from 3.3 to 4.5. The predicted charge of CsgA at pH 7.4 was −6.3, and it increased by several folds for the five other β-solenoid proteins, with the highest charge of −116.6 observed for El43-CsgA. The hydrophobicity, as predicted by their Gravy score (−0.7 to −0.1) indicated that all five de novo β-solenoid proteins are more hydrophobic than CsgA (FIG. 10).

TABLE 1
Amino Acid Composition of the β-Solenoid Proteins

| Amino acid | | | | | | |
|---|---|---|---|---|---|---|
| Residue % | CsgA | Hs13-CsgA | Am18-CsgA | Bc36-CsgA | El43-CsgA | Er46-CsgA |
| G | 21.37 | 12.22 | 10.9 | 13.22 | 12.96 | 13.54 |
| N | 12.21 | 10.14 | 11.21 | 9.97 | 9.95 | 13.54 |
| S | 9.16 | 7.04 | 11.53 | 11.81 | 12.11 | 9.03 |
| Q | 8.4 | 4.97 | 9.97 | 8.99 | 9.95 | 8.52 |
| A | 7.63 | 8.28 | 8.1 | 10.73 | 7.23 | 13.2 |
| T | 6.87 | 6.42 | 6.85 | 13.76 | 11.08 | 8.09 |
| V | 6.11 | 10.77 | 7.79 | 8.88 | 8.54 | 6.13 |
| D | 6.11 | 8.7 | 7.17 | 7.37 | 9.58 | 5.28 |
| L | 4.58 | 6.42 | 4.98 | 2.6 | 4.51 | 5.45 |
| Y | 3.05 | 1.24 | 3.12 | 0.87 | 0 | 1.28 |
| I | 2.29 | 5.8 | 3.43 | 4.01 | 4.88 | 5.79 |
| F | 2.29 | 4.14 | 2.49 | 1.95 | 3.47 | 2.39 |
| H | 2.29 | 0.21 | 2.18 | 0 | 0.09 | 0.51 |
| P | 1.53 | 0.62 | 0.31 | 1.95 | 0.75 | 1.62 |
| K | 1.53 | 1.04 | 2.49 | 0.43 | 0.28 | 1.45 |
| R | 1.53 | 0.62 | 1.56 | 1.3 | 1.22 | 1.79 |
| E | 1.53 | 7.87 | 2.8 | 1.73 | 2.82 | 1.7 |
| M | 0.76 | 2.28 | 2.18 | 0.43 | 0.47 | 0.43 |
| W | 0.76 | 1.24 | 0.93 | 0 | 0.09 | 0.26 |
| C | 0 | 0 | 0 | 0 | 0 | 0 |

TABLE 2
Physiochemical Properties of β-Solenoids

| | | | Net | | | |
|---|---|---|---|---|---|---|
| | Molecular | | Charge | | | |
| | Weight | Isoelectric | at Ph 7.4 | | | |
| Protein | (kDa) | Point (Pi) | (Z) | Interpretation | SASA (A²) | Gravy |
| CsgA | 13.1 | 4.528 | −6.319 | Slightly acidic; minor | 13247.397 | −0.7183 |
| | | | | negative charge at pH | | |
| | | | | 7.4, moderately stable | | |
| | | | | and soluble. | | |
| Hs13- | 34.02 | 4.401 | −19.261 | Highly acidic; significantly | 20327.082 | −0.6374 |
| CsgA | | | | negative charge at pH | | |
| | | | | 7.4, highly soluble, may | | |
| | | | | strongly repel other | | |
| | | | | negatively charged | | |
| | | | | molecules. | | |
| Am18- | 50.59 | 3.358 | −72.658 | Slightly acidic; moderate | 15050.855 | −0.1058 |
| CsgA | | | | negative charge at pH | | |
| | | | | 7.4, stable and soluble. | | |
| Bc36- | 91.04 | 3.519 | −68.719 | Highly acidic; significantly | 37039.582 | −0.4354 |
| CsgA | | | | negative charge at pH | | |
| | | | | 7.4, highly soluble, may | | |
| | | | | strongly repel other | | |
| | | | | negatively charged | | |
| | | | | molecules. | | |
| El43- | 107.81 | 3.325 | −116.609 | Extremely acidic; very | 105950.086 | −0.4524 |
| CsgA | | | | high negative charge at | | |
| | | | | pH 7.4, extremely | | |
| | | | | soluble, strong repulsion | | |
| | | | | likely. | | |
| Er46- | 117.63 | 4.229 | −44.521 | Acidic; high negative | 117655.484 | −0.3599 |
| CsgA | | | | charge at pH 7.4, high | | |
| | | | | solubility and potential for | | |
| | | | | repulsive interactions. | | |

### Decoding β-Solenoid Protein Structure Stability

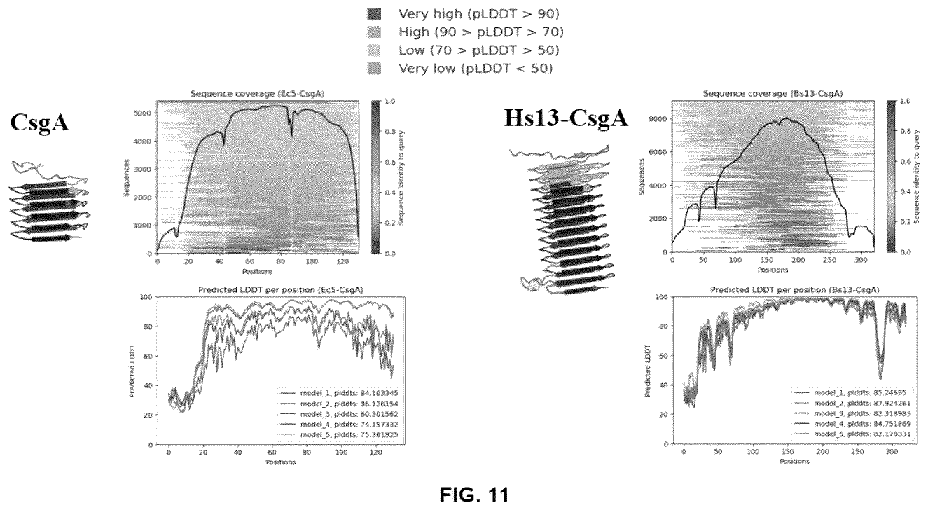

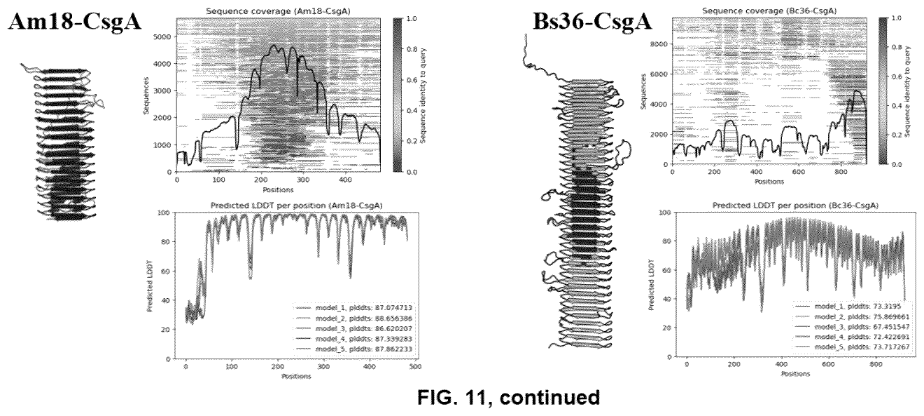

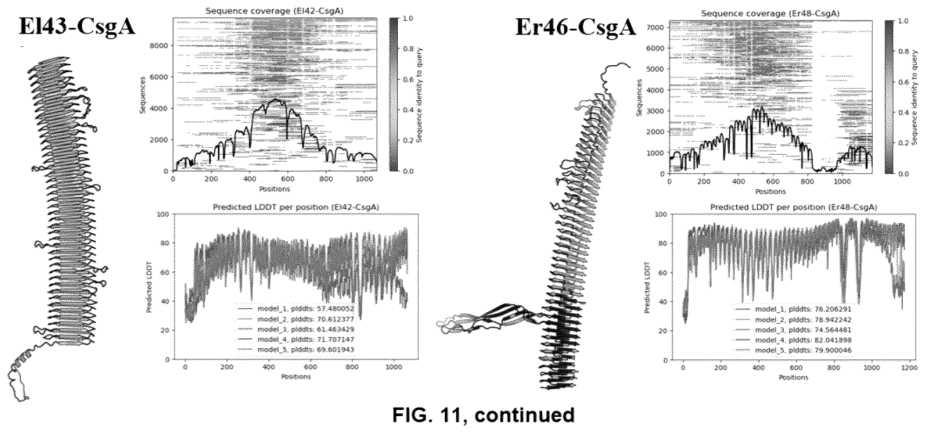

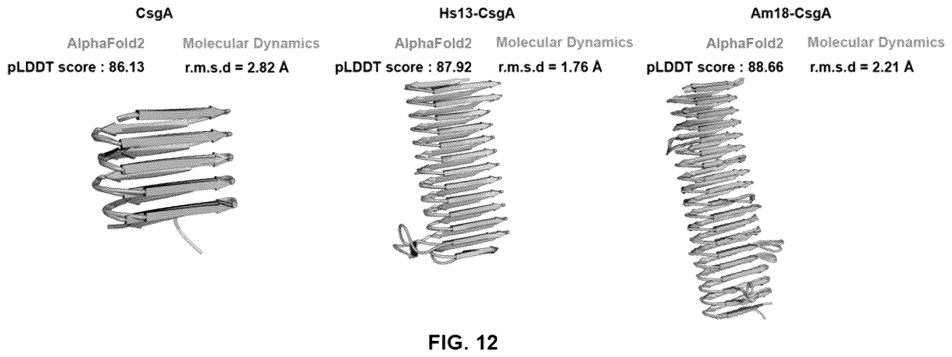

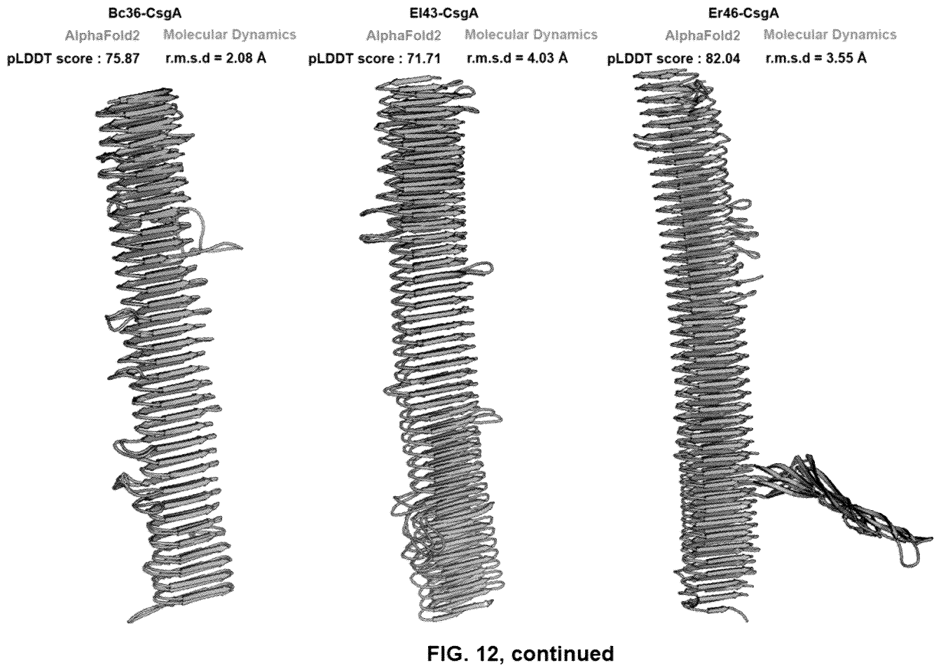

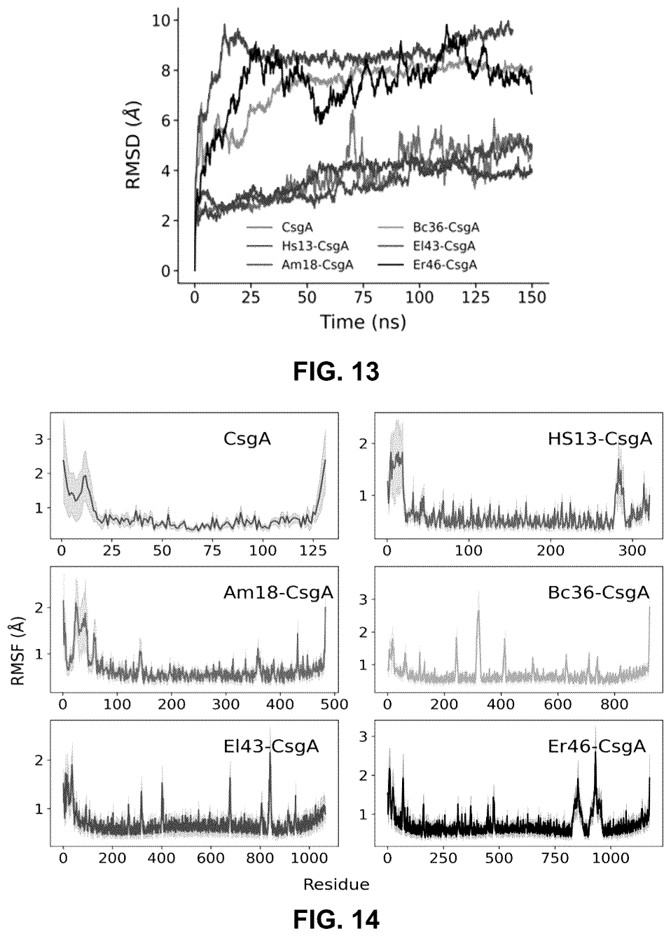

All six β-solenoid proteins showed pLDDT (predicted local distance difference test) confidence scores between 71 and 89, which indicated a good backbone prediction (FIG. 11). Using structures predicted by AlphaFold2, AA MD simulations were conducted to gain molecular-level insights into the stability of β-solenoid proteins, focusing on both structural and energetic analyses. For all six proteins, the root mean square deviation (RMSD) of all heavy atoms in backbone and the side chains of amino acids in β-sheets, increases with increasing residues (initial equilibration of less than ˜5 ns), and it remains stable during the remainder of the simulations run (FIG. 3E). RMSD of the entire protein showed slightly more perturbations and higher RMSD values, which could be due to the fluctuations in the N-terminal and outlier regions, which is consistent with the pLDDT scores (FIGS. 12-13). The residue-wise root mean squared fluctuations (RMSF) was consistent with the RMSD analysis, showing that the N-terminal and outlier residues had more fluctuations than the β-sheet region residues (FIG. 14).

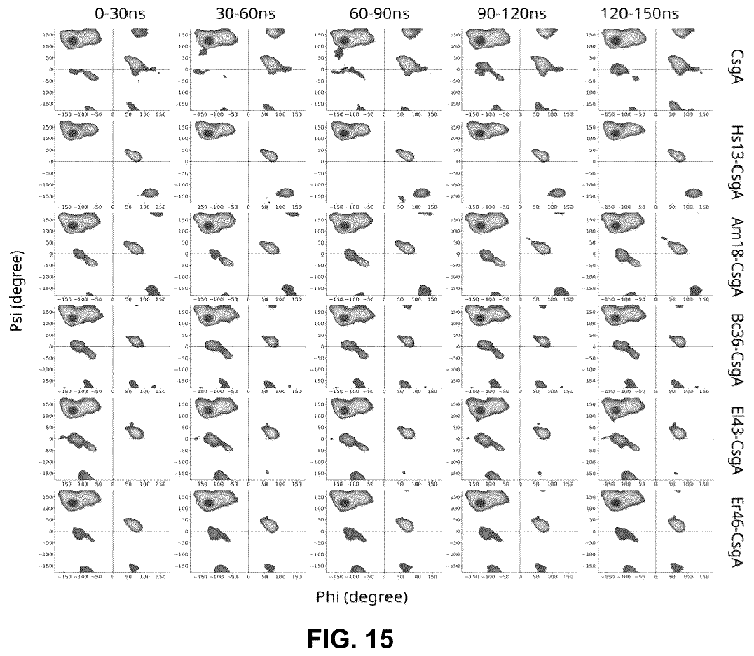

The Ramachandran plot showed a dense cluster of points with ϕ≈−120° and ψ=120° throughout 150 ns MD simulations, suggesting that the overall backbone dihedral angles are relatively preserved in the β-sheet region, with minor presence of α-sheets in all proteins (right α-sheet: ϕ=−60° and ψ=−45°; left α-sheet: ϕ=70° and ψ=35°; Table 3, FIG. 15). The hydrogen bond characteristics between all protein residue pairs forming hydrogen bonds were evaluated using a geometric criterion described in the Method section. The hydrogen bond analysis indicates that the presence of bifurcated hydrogen bonds, in addition to hydrogen bonds in β-sheets, may further contribute to the structural stability of the proteins. A slower decay of hydrogen bond autocorrelation function was observed for the five de novo β-solenoid proteins compared to CsgA, which indicates that the hydrogen bonds formed between amino acid pairs in the five de novo β-solenoid proteins are more stable compared to those in CsgA protein.

TABLE 3
α-sheet and β-turn Percentage in β-Solenoid Proteins

| Variants | α-sheet | β-turn |
|---|---|---|
| CsgA | 3.37 | 18.69 |
| Hs13-CsgA | 2.90 | 13.58 |
| Am18-CsgA | 5.75 | 21.39 |
| Bc36-CsgA | 3.08 | 19.97 |
| El43-CsgA | 3.99 | 18.69 |
| Er46-CsgA | 3.13 | 15.88 |

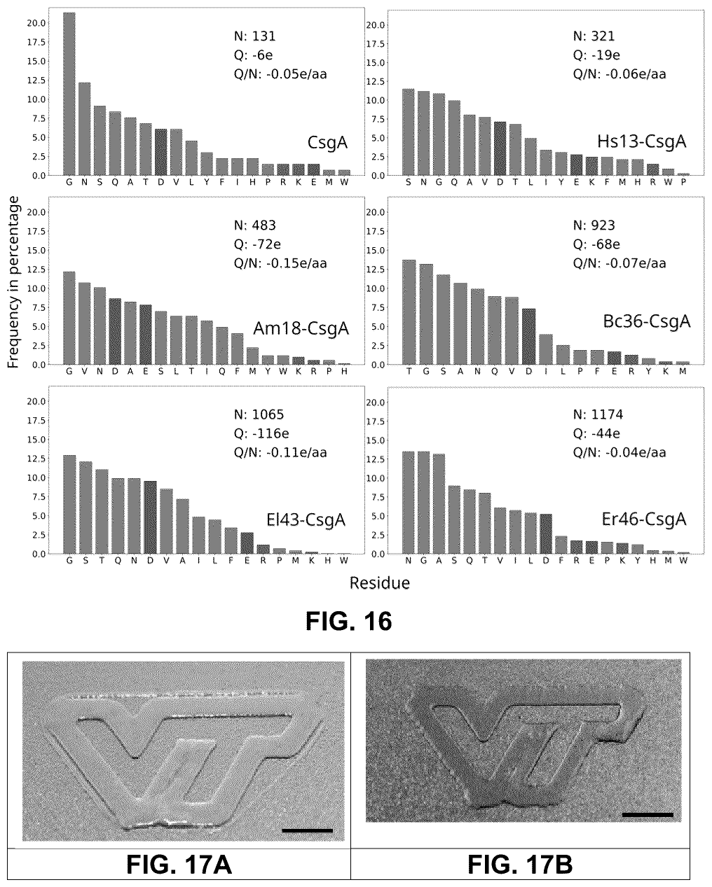

A detailed structural analysis revealed that the five β-solenoid proteins contain a higher number of charged residues (e.g., D, E, K, R) compared to CsgA (FIG. 16). Additionally, the local environment of each residue (within 12 Å) is predominantly composed of glycine (G) and polar residues (e.g., N, Q, S, T) rather than non-polar residues (e.g., I, L, M, F, W). To investigate further the role of charged residues and their local environments, averaged non-bonded interaction energies (IE) were calculated between KR and KR (IE1), between KR and all residues (IE2) and between non-KR and all residues (IE3) (FIG. 3G). Across all six proteins, the electrostatic interactions in IE1, IE2, and IE3 were generally more favorable (more negative) compared to van der Waals interactions but exhibited varying patterns. The electrostatic interactions in IE1 were consistent across most proteins, except for Bc36-CsgA, which showed less favorable interactions (less negative). In contrast, electrostatic interactions in IE2 and IE3 displayed greater variability, with CsgA and Er46-CsgA showing more favorable energies than the other proteins. Notably, IE2 electrostatic energies were more favorable than IE3. These results highlight that both charge placement and the local environment of these residues play an important role in determining electrostatic energies. Meanwhile, van-der Waals energy contributions in IE1, IE2, and IE3 were distinct but consistent across all six proteins, likely due to their similar geometries. Overall, MD simulations revealed that all six β-solenoid proteins are stabilized by hydrogen bond formation among the β-sheets and favorable electrostatic interactions between the key residues and all residues.

### Biomanufacturing of Modular De Novo Autogenic Engineered Living Materials

The above described five de novo CsgA homologs are from extremophilic non-model bacteria, which poses several challenges, such as culturing bacteria, scalable production, and genetic modifications. To circumvent these challenges, the curli secretion machinery of E. coli has been modified to develop a platform termed SECRETE, which stands for Secretion of Extra Cellular REcombinant β-solenoid proteins for Tailorable Engineered living materials. Herein, E. coli strain PQN4, wherein the curli operon is deleted, was used, which makes it suitable for curli overproduction. These de novo β-solenoid protein monomers were designed by integrating Sec (N-terminal signal sequence; SEQ ID NO. 17) and N22 (N-terminal curli-specific targeting sequence; SEQ ID NO. 18) sequences of E. coli to facilitate extracellular secretion and self-assembly.

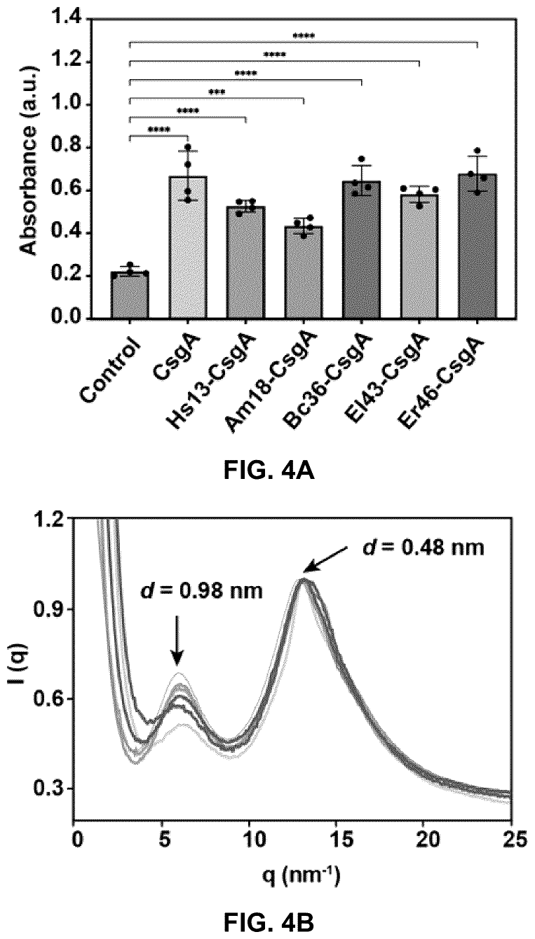

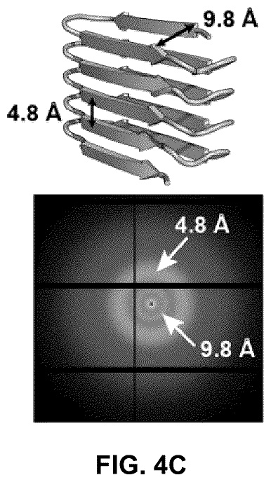

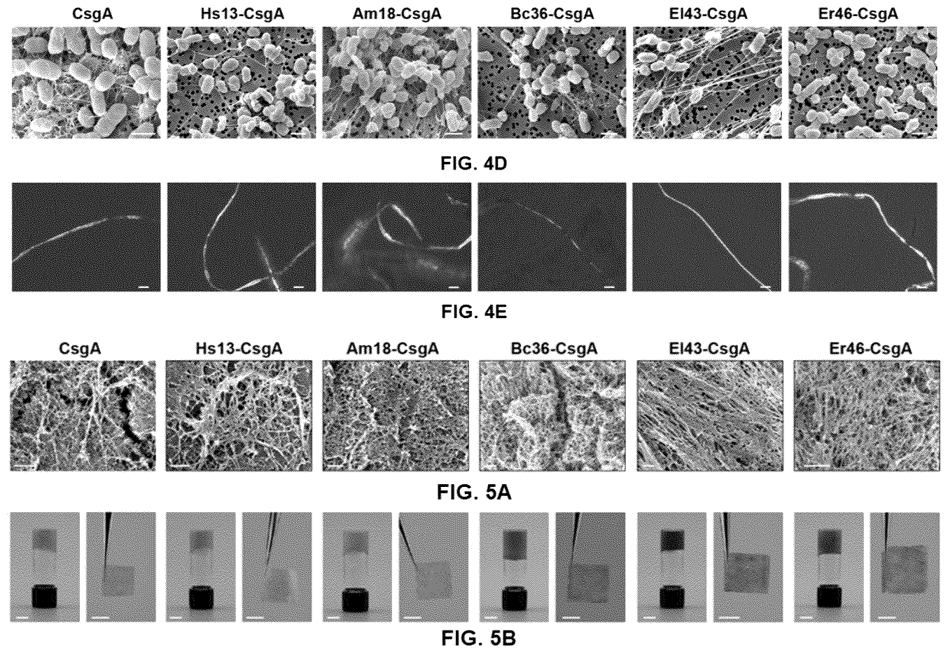

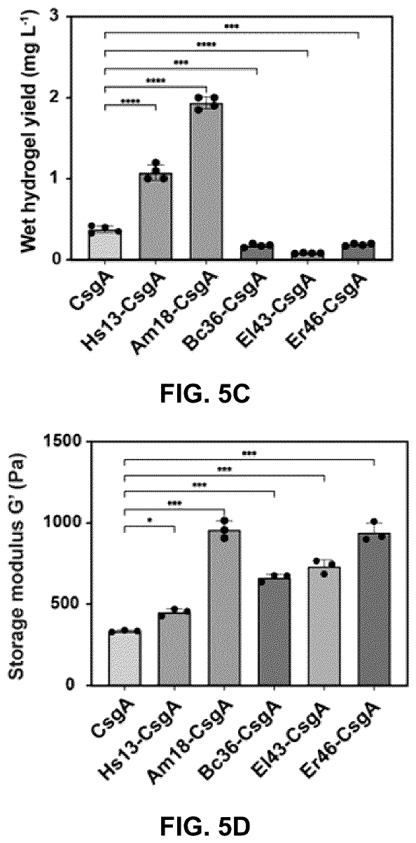

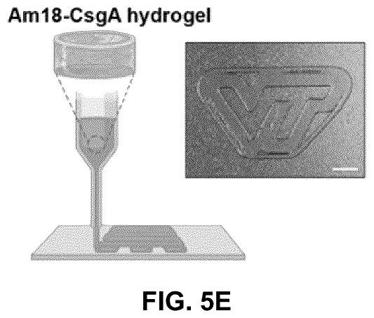

Congo Red dye assay was utilized to experimentally and qualitatively assess the cross-β structure of Hs13-CsgA, Am18-CsgA, Bc36-CsgA, El43-CsgA, and Er46-CsgA (FIG. 4A). All these five CsgA homologs showed good CR-binding like that of CsgA; the relative differences in the Congo red binding could be attributed to slightly different 1) affinities due to their distinct amino acid compositions and structure and 2) levels of production of CsgA homologs. Wide-angle X-ray scattering (WAXS) analysis revealed characteristie d-spacing (interplanar spacing) values of 0.98 nm and 0.46 nm, corresponding to inter-β-sheet and inter-β-strand distances, respectively, consistent with that of CsgA, indicating the cross-β architecture of CsgA homologs (FIGS. 4B-4C). Further, ultrastructural characterization of bacterial cultures by field-emission scanning electron microscopy (FESEM) showed the protein nanofibers self-assembled in the extracellular milieu (FIG. 4D). Moreover, the characteristic birefringent property like CsgA was observed for all CsgA homolog nanofibers, under crossed polarizers (FIG. 4E). Subsequently, to demonstrate the biomanufacturing of macroscopic modular ELMs, hydrogels of all six variants were produced by using the filtration protocol, and the FESEM images showed the dense network of nanofibers in the hydrogels (FIG. 5A). In addition, the hydrogels were cast on templates and dried under ambient conditions to fabricate aquaplastics, which could find potential applications as biodegradable bioplastics, robust coatings, and sustainable materials (FIG. 5B).

The yield of the hydrogels varied, Am18-CsgA (1938±75 mg L⁻¹) and Bs13-CsgA (1075 ±96 mg L⁻¹) showed the highest yields, both exceeding that of E. coli CsgA (375±42 mg L⁻¹), making them more suitable for scalable biomaterial production (FIG. 5C). As the five de novo β-solenoid proteins have more cross-β repeats than CsgA and the nanofibers network observed in the FESEM images were slightly different, it was anticipated that their mechanical properties could be different. To test this, the storage modulus (G′) of hydrogels was investigated, which revealed that all five de novo-solenoids exhibited significantly higher G′ values than CsgA. Interestingly, G′ was found to increase (by two folds) with the number of cross-β repeats (from 5 to 46). However, G′ of Am18-CsgA with 18 repeats was also the same as that of Er46-CsgA with 46 repeats, which could be attributed to differences in the 1) physicochemical properties such as greater hydrophobicity and lower charge, 2) aggregation of nanofibers and in turn the interactions with water that facilitates gelation (FIG. 5D). In addition, these curli hydrogels are suitable for extrusion bioprinting due to their shear-thinning behavior, a key property that enables it to flow like a fluid under the influence of external stress and upon removal of the external stress, it reverts to its initial viscosity. Building on this property, the 3D printability of de novo β-solenoid protein hydrogels was demonstrated by printing the Virginia Tech University logo using Am18-CsgA, which was utilized due to the higher yield of hydrogel (FIGS. 5E and 17A-17B).

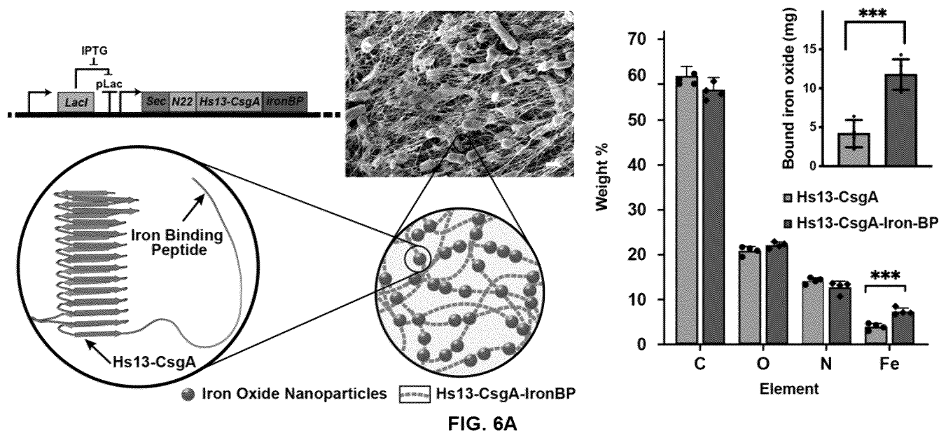

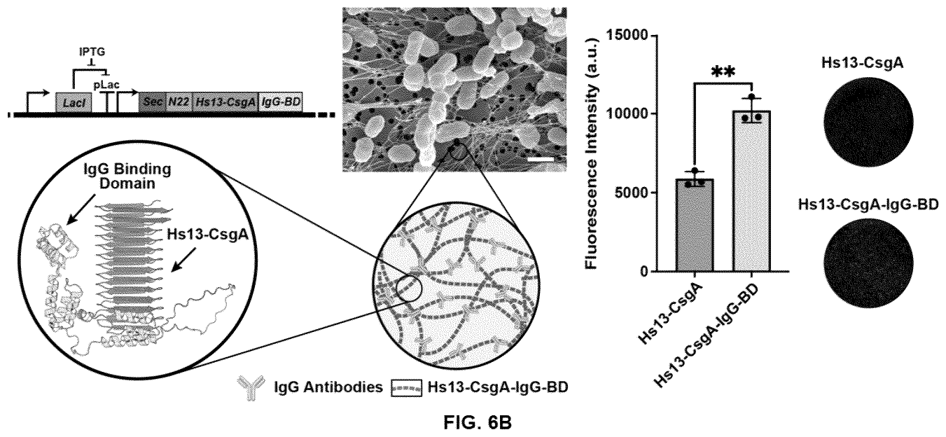

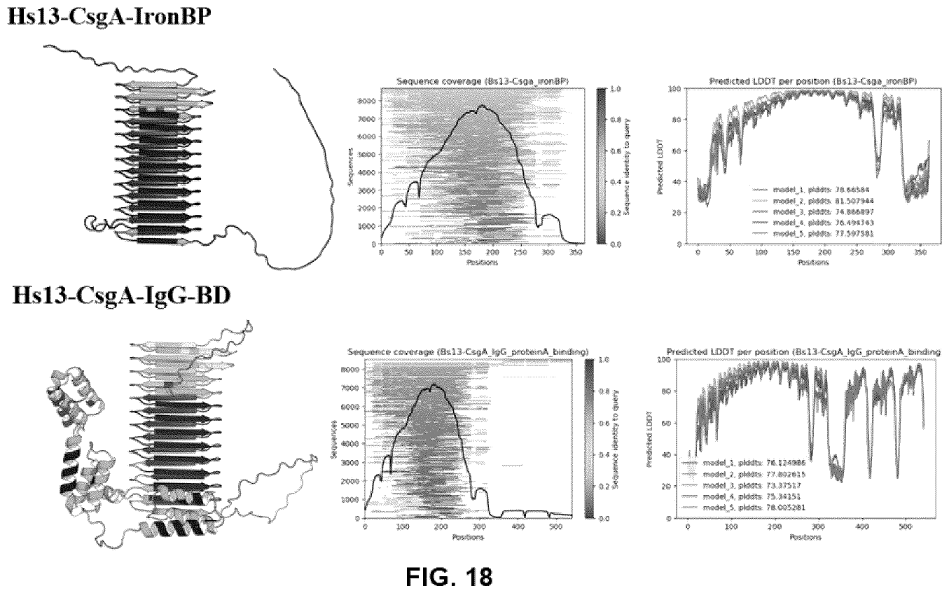

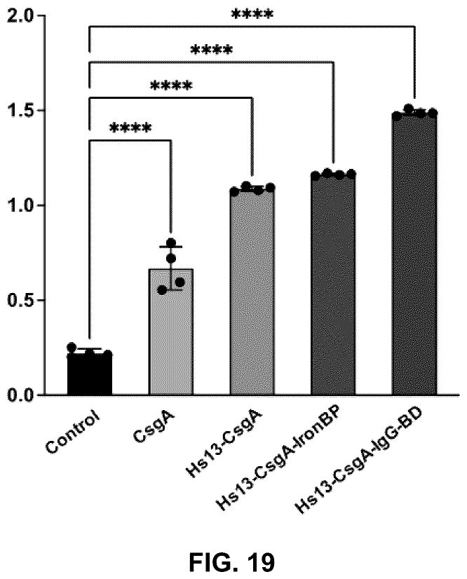

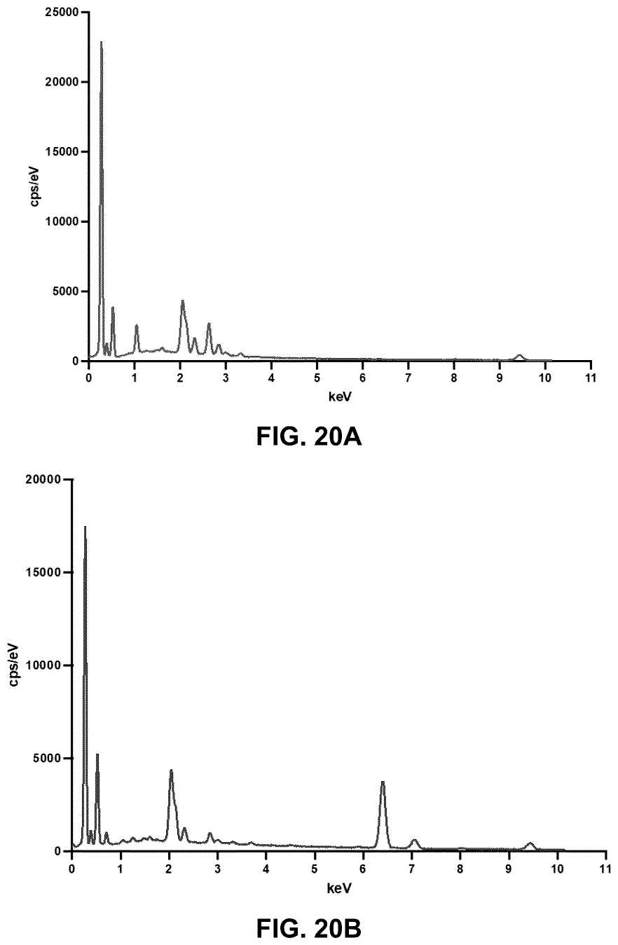

Having demonstrated the modulation of physicochemical properties of de novo β-solenoid protein building block-based nanofibers and macroscopic materials, their potential for programming the biological functionalities was next explored. To achieve this, Hs13-CsgA was selected as the β-solenoid protein building block, which was genetically fused with a functional peptide or protein to assess whether the engineered variants could be secreted and self-assembled into biofunctional nanofibers. Herein, the C-terminal of Hs13-CsgA was genetically fused with 8-amino-acid iron-binding peptide (Hs13-CsgA-IronBP) or 185-amino-acid Immunoglobulin G (IgG) antibody binding protein (Hs13-CsgA-IgG-BD) via a 36-amino-acid flexible linker. AlphaFold2 structure predictions confirmed that both Hs13-CsgA-IronBP and Hs13-CsgA-IgG-BD retained their characteristic 13 repeats β-solenoid architecture with no structural disturbances caused by the fused peptide or protein domain (FIGS. 6A-6B and 18). Congo red assay and FESEM images of Hs13-CsgA-IronBP and Hs13-CsgA-IgG-BD confirmed their cross-β structure and nanofibrillar assemblies, respectively (FIG. 19). The iron-binding assay showed that Hs13-CsgA-IronBP had a significantly higher weight of bound iron oxide nanoparticles than the wildtype Hs13-CsgA nanofibers. The elemental composition studied by using energy dispersive X-ray analysis (EDAX) also confirmed that Hs13-CsgA-IronBP has significantly higher iron content (FIGS. 6A and 20A-20). Similarly, Hs13-CsgA-IgG-BD was found to bind red fluorescent IgG antibodies nearly twice higher than wildtype Hs13-CsgA nanofibers, as indicated by fluorescence intensity measurements and imaging (FIG. 6B). These results highlight the utility of de novo β-solenoid nanofibers to tailor their functionalities for biomining, bioremediation, biosensing, and biomedical applications.

### Example 2: Discussion

There are only a few examples of functional amyloids, and therefore, it was initially surprising to find that 33,564 proteins listed in the NCBI database are homologs of CsgA. Interestingly, recent work has reported that 22% (43279) of the genomes out of the 201210 bacterial genomes searched contained one or more predicted CsgA sequences with one or more curli repeat signatures. Thus, the protein mining results corroborate closely with the previously reported genome mining results and, on the other hand, emphasize the enormous scale to which CsgA-like proteins are prevalent in the natural world. Moreover, the curli system is not exclusively limited to E. coli, and ˜33% of identified CsgA homologs are secreted by bacteria phylogenetically spread across at least four major bacterial phyla, each with its own distinct curli system. This evolutionary diversity and abundance of functional proteins highlight the untapped potential that could be explored and exploited for advanced autogenic ELMs.

The rapid advancement of AI-driven structure prediction tools such as AlphaFold2 has revolutionized the ability to explore the structural properties of diverse proteins. These tools have significantly accelerated the design, screening, and development of novel functional protein-based materials. As cryo-TEM (transmission electron microscopy) and single crystal X-ray diffraction studies of CsgA and its homologs have proven extremely difficult, structure prediction tools like AlphaFold2 are beneficial in getting critical insights. MD simulations provide valuable insights, demonstrating the critical role of KRs in stabilizing β-solenoid structure. However, the evolutionary diversity observed in de novo analogs indicates that even non-conserved KR₁ and KR₇ residues lead to stable structures. Thus, it opens a broad landscape for tinkering and rationally designing de novo functional protein materials.

In summary, herein it has been demonstrated that E. coli curli machinery could be meticulously utilized to produce de novo autogenic ELMs by employing β-solenoid protein building blocks of non-model extremophilic bacteria. Despite having distinct amino acid compositions and molecular weights (up to 9 times of native CsgA), their biosynthesis, secretion, and extracellular self-assembly into stable β-solenoid protein nanofibers highlight the robustness and versatility of the SECRETE platform to biomanufacture de novo autogenic ELMs. Moreover, the variations in the yields of hydrogels (375 to 1938 mg L⁻¹) and storage modulus (335 to 959 Pa) might be influenced by the physicochemical properties of β-solenoid building blocks, such as the number of β-sheet repeats, hydrophobicity, charge, and amino acid compositions, which needs further investigation. The fabrication of hydrogels, films, and well-defined 3D architectures with programmable physical, chemical, and biological functionalities will open Pandora's box to rationally design sophisticated de novo autogenic ELMs for biomedical and environmental applications.

### Example 3: Materials and Methods

### Cell Strains and Plasmids

All experiments were conducted using PQN4, E. coli strain derived from LSR10 (MC4100, ΔcsgA, λ(DE3), CamR) with the deletion of curli operon (ΔcsgBACEFG). The genes encoding CsgA and its 5 homologs and the two engineered domains were synthesized by Twist BioScience and cloned into the pET21d vector using overlap extension and isothermal Gibson Assembly (New England Biolabs). The whole plasmid sequences were confirmed by Plasmidsaurus. These plasmids also included genes encoding proteins essential for curli biosynthesis, such as csgC, csgE, csgF, and csgG. Five different β-solenoid proteins, namely, Hs13-CsgA, Am18-CsgA, Bc36-CsgA, El43-CsgA, and Er46-CsgA were inserted in place of the E. coli wild-type CsgA after the SEC (N-terminal signal sequence) and the N22 (N-terminal curli-specific targeting sequence) to facilitate the secretion of CsgA variants into the extracellular space. Additionally, two engineered β-solenoid protein variants were created, namely, Hs13-CsgA-IronBP and Hs13-CsgA-IgG-BD by fusing iron-binding peptide (IronBP) and antibody IgG binding domain (IgG-BD) to the C-terminus of Hs13-CsgA via a 36-amino-acid flexible linker. All gene sequences and protein sequences used in this study are provided in the sequence listing.

### Protein Sequence Mining and AlphaFold2 Modeling of CsgA and its Homologs

The protein structure database of the National Center for Biotechnology Information (NCBI) was used and the search query-Major [Organism] OR major [All Fields] AND curlin [All Fields]—was employed to identify CsgA homologs. This search yielded 33,564 entries, annotated as having the curlin subunit CsgA, either through computational analysis or by identifying sequences using a regular expression based on the minimalistic curli repeat (X₆QXGX₂NX₁₀) described by Chapman et al. The lengths of the resulting protein sequences varied from 141 to 1390 amino acids. From these 33,564 entries, 50 sequences were randomly selected, and to predict their 3D structure, AlphaFold2 (version 2.1.1) was used on the Tinkercliffs HPC cluster at the Advanced Research Computing Center, Virginia Tech, utilizing NVIDIA A100-80G architecture. Structural images were generated using PyMOL (version 1.16). Batch protein structure predictions were performed, running for six recycles and generating five models per sequence. The models were ranked using the pLDDT score, with the highest-ranked model being used for subsequent analyses. Out of these 50 sequences, 5 sequences were selected, having lengths across the whole spectrum of 141 to 1390 amino acids. Genes of these 5 sequences were synthesized to validate their production in the E. coli PQN4 strain experimentally. These variants were named based on the first letter of the species, followed by the number of cross-β amyloid repeats and CsgA—to indicate that it is a homolog. For example, the CsgA homolog of Halomonas saliphila with 13 cross-β amyloid repeats was named Hs13-CsgA. Similarly, 18, 36, 43, and 46 cross-β amyloid repeats of CsgA homologs of Alteromonas macleodii, Blastomonas sp. CACIA14H2, Erythrobacter longus, and Ensifer sp. Root31 were labelled as Am18-CsgA, Bc36-CsgA, El43-CsgA and Er46-CsgA, respectively. In addition, two engineered variants, Hs13-CsgA-IronBP and Hs13-CsgA-IgG-BD, were also predicted in a similar fashion.

### Molecular Dynamics (MD) Simulation

All-atom MD simulations of the six β-solenoid proteins—CsgA, Hs13-CsgA, Am18-CsgA, Bc36-CsgA, El43-CsgA, and Er46-CsgA in explicit water were conducted using the NAMD-2.14 software. The initial structures were obtained from the AlphaFold2 server using the experimental FASTA sequences. These β-solenoid proteins were solvated using TIP3P water in a cubic box with a minimum of 15 Å padding in each direction. The solvated β-solenoid proteins were neutralized by adding sodium ions to balance the net charges of these proteins (FIG. 12). The atomistic MD simulations were performed in 2 fs timestep in the NPT ensemble with the periodic boundary conditions applied in all directions. The Charmm-36m (July 22) force field (FF) was used to model the bonded and non-bonded interactions. The standard 12-6 Lennard-Jones potential was applied with a 12 Å cutoff, and the Particle Mesh Ewald (PME) method was used to calculate the long-range electrostatic interactions. The Langevin thermostat and Parrinello-Rahman barostat were implemented to maintain a temperature at 300 K and pressure at 1.01325 bar, respectively. This simulation setup maintained the system stability while accommodating fluctuations in density. Energy minimization of 50,000 steps was followed by 150 ns MD simulations, in which the last 50 ns were treated as a production run. The trajectory data was stored in 1 ps intervals during these MD simulations. All the analyses were carried out using the last 50 ns production run except for the RMSDs, which were calculated for the entire 150 ns trajectory to compare and estimate the structural changes from the initial AlphaFold2 generated structures.

### Analysis Method of MD Simulation

The RMSD and RMSF of the amyloids were calculated using the VMD software package, and the MD Analysis package was used to calculate the hydrogen bond autocorrelations and Ramachandran plots. The hydrogen bond criteria used were a donor-hydrogen distance cutoff of 1.2 Å, a donor-acceptor distance cutoff of 3.0 Å, and a donor-hydrogen-acceptor angle cutoff of 150°. An automated tool utilizing the NAMD executable to calculate the residue-residue interaction energies for the large β-solenoid protein variants was developed. These energies were calculated based on the non-bonded interactions, including electrostatic and van der Waals contributions. Water and ions were removed from the system to calculate the energies. The last 50 ns trajectory is analyzed with a 0.1 ns stride. A percent cutoff of 60% and 12 Å filtering distance were applied, meaning that only pairs of residues whose center-of-mass come closer than 12 Å in at least 60 percent of trajectory frames were included in the non-bonded energy calculations. The energy data was verified with the gRINN software package.

### Microbial Production of β-Solenoid Protein Nanofibers

All plasmids (CsgA, Hs13-CsgA, Am18-CsgA, Bc36-CsgA, El43-CsgA, Er46-CsgA, Hs13-CsgA-IronBP, and Hs13-CsgA-IgG-BD) were transformed into the E. coli strain PQN4. The transformed cells were streaked onto lysogeny broth (LB) agar plates containing 100 μg mL-1 carbenicillin and 0.5% glucose (w v⁻¹) for catabolite repression of T7RNAP and incubated overnight at 37° C. A single colony from each plate was picked and separately cultured at 37° C. in 5 mL of LB media with 100 μg mL-1 carbenicillin and 2% glucose (w v⁻¹). These overnight cultures (PQN4_CsgA, PQN4_Hs13-CsgA, PQN4_Am18-CsgA, PQN4_Bc36-CsgA, PQN4_EI43-CsgA, PQN4_Er46-CsgA, PQN4_Hs13-CsgA-IronBP, and PQN4_Hs13-CsgA-IgG-BD) were then transferred to fresh 500 mL LB media containing 100 g mL-1 carbenicillin. The cultures were incubated in shaking incubators (225 rpm, 37° C.) for 48 hours to achieve expression of β-solenoid proteins and assembly into functional amyloid nanofibers. As a negative control, PQN4 was transformed with a sham (all curli genes, including csgA were absent), and pET21d plasmid was used.

### Quantitative Congo Red Dye Binding Assay

One milliliter of bacterial culture (48 h, 500 mL) was centrifuged at 6000 rpm for 10 minutes. The resulting cell pellet was resuspended in a 0.025 mM solution of Congo red in phosphate-buffered saline (PBS) and incubated for 10 minutes. The cells were then pelleted again at 14,000 rpm for 10 minutes, and the absorbance of the supernatant (200 pl) at 490 nm was measured using a microplate reader. This absorbance value was subtracted from 0.025 mM Congo red in PBS, which was subsequently normalized by the OD600 of the original bacterial culture to quantify the production of functional amyloid nanofibers. The assay was performed in four biological replicates.

### Congo Red Birefringence

Functional amyloid nanofibers were stained with Congo red and evaluated for birefringence. One milliliter of bacterial culture was centrifuged at 6000 rpm for 10 minutes. The resulting pellet was resuspended in 10 μL of 500 μM Congo red solution in 80% ethanol and incubated for 60 minutes at room temperature. XploRA™ PLUS Confocal Raman Microscope, equipped with an inverted camera and crossed polarizing light filters, was utilized to visualize and assess birefringence.

### Preparation of β-Solenoid Protein Hydrogels

E. coli PQN4 strain with the desired plasmid was cultured in 500 mL of LB media in a shaking incubator at 37° C., 225 rpm for 48 hours to produce the β-solenoid protein nanofibers. The resulting 500 mL culture was treated with urea to a final concentration of 0.8 M and kept at 4° C. for 1 h. The treated cultures were then concentrated by vacuum filtration through a 90-mm diameter polycarbonate membrane with 10-μm pores (EMD Millipore). The resulting concentrated biofilm (nanofibers) was washed thrice with 50 mL of sterile deionized (DI) water on the filter membrane. The biofilm was subsequently incubated with 100 mL of 8 M urea solution in water for 5 minutes, followed by vacuum filtration and washing with 200 mL of DI water to remove the bacterial debris lysate. The resulting biomass on the filter membrane was then treated with 50 mL of 5% (w v⁻¹) SDS (sodium dodecyl sulfate) solution (gelator/plasticizer) in water for 5 minutes, followed by vacuum filtration and an additional wash with 500 mL of DI water. The β-solenoid protein hydrogel formed on the filter membrane was then collected and stored at 4° C.

Fabrication of Aquaplastic from β-Solenoid Protein Nanofibers

To fabricate 2D aquaplastic films, β-solenoid protein nanofibers based hydrogels were cast onto a flat, flexible substrate such as plastic wrap or aluminum foil. These were left to dry overnight under ambient conditions to form aquaplastic films of lateral dimensions 1 cm*1 cm. Once thoroughly dried, the aquaplastic film was gently peeled off the substrate and stored at room temperature.

### Field-Emission Scanning Electron Microscopy Sample Preparation and Imaging

100 μl of the E. coli cultures or 10 mg of hydrogel samples were filtered/spread onto polycarbonate membranes with a 0.22 μm pore size under vacuum and then placed in a fixative solution containing 1 mL of 4% glutaraldehyde and 1 mL of 4% paraformaldehyde buffer for 2 hours at room temperature. After fixation, the membranes were gently rinsed with water and subjected to a series of 200-proof ethanol washes with increasing concentrations (25%, 50%, 75%, 100%, and 100% v v⁻¹), each for 15 minutes. The samples were then processed using a critical point dryer. The dried membranes were mounted on Scanning Electron Microscopy sample holders with carbon adhesives and coated with a 10 to 20 nm layer of Pt/Pd. Images were captured using a JEOL IT500 SEM equipped with a field-emission gun operating at 5-10 kV.

### Wide-Angle X-Ray Scattering

Wide-angle X-ray scattering (WAXS) experiments on the aquaplastics were performed using a Xenocs Xeuss 3.0 SAXS/WAXS equipped with a GeniX 3D Cu HFVLF microfocus X-ray source utilizing Cu Kα radiation (λ=0.154 nm). The sample-to-detector distance was 42 mm, and the q-range was calibrated using a Lanthanum hexaboride standard. Two-dimensional scattering patterns were obtained using a Dectris EIGER 4M detector with an exposure time of 30 minutes. Data reduction was performed using XSACT software provided by Xenocs.

### Rheology Studies of the Hydrogels

The viscoelastic properties of the β-solenoid protein hydrogels were assessed using a μ-volume sample holder with the ElastoSens™ Bio (Rheolution, Montreal, Canada). 250 μg gel sample was cast into the sample holder. The storage modulus (G′, Pa) of each sample was measured to evaluate their viscoelastic characteristics. Each test was performed in technical duplicates for three biological replicates, resulting in a total of six readings per time point. G′ was recorded every 5 seconds over a 1-minute period.

### 3D Printing of β-Solenoid Protein Hydrogels (Microbial Ink)

The bioinks were transferred into a 10-mL Luer-Lok™ syringe and centrifuged at 112×g for 2 minutes to remove any air bubbles. Bioprinting was conducted at room temperature using a Hyrel HYDRA 21 Bioprinter (Hyrel3D, Norcross, GA, USA) equipped with a 10-ml syringe dispensing head (SDS-10) for gel printing. 22G and 20G needles with a premade 0.25-inch blunt end were utilized. The printing speed of 2 mm s⁻¹ and the blunt needle diameters of 0.41 mm (22G) and 0.60 mm (20G) were selected after optimization. This process involved assessing various printing speeds and needle sizes with internal diameters ranging from 0.26 to 0.60 mm to achieve optimal pattern fidelity, reproducibility, and consistency across different polymer concentrations and viscosities of the amyloid hydrogel inks. Initial bioprinting trials were performed at feed rates ranging from 2 to 10 mm s⁻¹. The 3D STL files were prepared using the slic3r engine integrated with the Hyrel HYDRA 21 Bioprinter software.

### Optical Images

Optical images were obtained using a Canon EOS rebel T7 DSLR Camera.

### Iron Oxide Binding Analysis by Genetically Engineered β-Solenoid Protein

The plasmid pET21d-Hs13-CsgA-IronBP and pET21d-Hs13-CsgA were separately transformed into PQN4 cells to produce engineered and non-engineered functional amyloid nanofibers. 50 mL of culture with expressed nanofibers was spun down, and the supernatant was discarded. This cell pellet was added with 5 mg of iron (III) oxide (<50 nm, Sigma-Aldrich) in 50 mL of water. Cell pellets with nanofibers were incubated at ambient temperature with light agitation for 2 hours to facilitate binding of iron oxide nanoparticles to nanofibers. Next, the entire mixture was deposited on a 47-mm diameter polycarbonate membrane with 10-μm pores (EMD Millipore) and washed with water several times to remove unbound iron oxide nanoparticles. The weight of iron oxide nanoparticles bound to Hs13-CsgA-IronBP was determined and compared to control samples with non-engineered nanofibers of Hs13-CsgA. Additionally, the engineered and non-engineered nanofibers with bound iron oxide nanoparticles deposited on the filter membrane were tested by energy-dispersive X-ray analysis (EDAX) to obtain elemental composition.

### Antibody Binding Analysis of Genetically Engineered β-Solenoid Protein

The plasmid pET21d-Hs13-CsgA-IgG-BD was transformed into PQN4 cells to express the engineered functional amyloids containing IgG binding protein fusion. The expressed engineered nanofibers were deposited on the filter membrane (dry weight of nanofibers 1.875±0.62 mg) and incubated with a solution of antibody Goat anti-Rabbit IgG with Alexa Fluor™ 594 (Thermo Fisher Scientific) for 2 hours. Next, filter membranes with deposited, engineered (Hs13-CsgA-IgG-BD) and non-engineered nanofibers (control, Hs13-CsgA) were washed with water several times to remove unbound antibodies. The bound antibodies were detected by fluorescence intensity of Alexa Fluor 594 using ChemiDoc Imaging System (BIO-RAD). Images were analyzed by utilizing ImageJ software.

### Statistics and Reproducibility

All experiments presented in this article were repeated at least three times (n=3) on biological replicates or distinct samples, and they are clearly stated in the figure captions and relevant method sections. All data are presented as the mean and standard deviation. All plotting and statistical analysis (ordinary one-way ANOVA or Welch t-test) were performed using GraphPad Prism 10 software.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. An, B. et al. Engineered Living Materials For Sustainability. Chem Rev 123, 2349-2419 (2023).
2. Barran-Berdon, A. L. et al. Enhanced purification coupled with biophysical analyses shows cross-beta structure as a core building block for Streptococcus mutans functional amyloids. Sci Rep 10, 5138 (2020).
3. Burns, N., et al. 3D extrusion bioprinting of microbial inks for biomedical applications. Adv Drug Deliv Rev 217, 115505 (2024).
4. Cao, Y. et al. Programmable assembly of pressure sensors using pattern-forming bacteria. Nature Biotechnology 35, 1087-1093 (2017).
5. Caro-Astorga, J., et al. Bacterial cellulose spheroids as building blocks for 3D and patterned living materials and for regeneration. Nat Commun 12, 5027 (2021).
6. Chapman, M. R. et al. Role of Escherichia coli curli operons in directing amyloid fiber formation. Science 295, 851-855 (2002).
7. Chen, A. Y. et al. Synthesis and patterning of tunable multiscale materials with engineered cells. Nat Mater 13, 515-523 (2014).
8. Costan, S. A., et al. Biophysical characterization of synthetic adhesins for predicting and tuning engineered living material properties. Matter 7, 2125-2143 (2024).
9. Cui, M. et al. Modular genetic design of multi-domain functional amyloids: insights into self-assembly and functional properties. Chem Sci 10, 4004-4014 (2019).
10. Darden, T., et al. Particle mesh Ewald: An N.log (N) method for Ewald sums in large systems. The Journal of Chemical Physics 98, 10089-10092 (1993).
11. Dorval Courchesne, N. M., et al. Scalable Production of Genetically Engineered Nanofibrous Macroscopic Materials via Filtration. ACS Biomater Sci Eng 3, 733-741 (2017).
12. Dueholm, M. S., et al. Curli functional amyloid systems are phylogenetically widespread and display large diversity in operon and protein structure. PLOS One 7, e51274 (2012).
13. Dunbar, M., et al. Dimerization energetics of curli fiber subunits CsgA and CsgB. npj Computational Materials 5, 27 (2019).
14. Duraj-Thatte, A. M. et al. Genetically Programmable Self-Regenerating Bacterial Hydrogels. Adv Mater 31, e1901826 (2019).
15. Duraj-Thatte, A. M. et al. Modulating bacterial and gut mucosal interactions with engineered biofilm matrix proteins. Sci Rep 8, 3475 (2018).
16. Duraj-Thatte, A. M. et al. Programmable microbial ink for 3D printing of living materials produced from genetically engineered protein nanofibers. Nat Commun 12, 6600 (2021).
17. Duraj-Thatte, A. M. et al. Water-processable, biodegradable and coatable aquaplastic from engineered biofilms. Nat Chem Biol 17, 732-738 (2021).
18. Elsacker, E., et al. Fungal Engineered Living Materials: The Viability of Pure Mycelium Materials with Self-Healing Functionalities. Advanced Functional Materials 33, 2301875 (2023).
19. Evans, M. L et al. Curli biogenesis: order out of disorder. Biochim Biophys Acta 1843, 1551-1558 (2014).
20. Gantenbein, S. et al. Three-dimensional printing of mycelium hydrogels into living complex materials. Nature Materials 22, 128-134 (2023).
21. Gilbert, C. et al. Living materials with programmable functionalities grown from engineered microbial co-cultures. Nat Mater 20, 691-700 (2021).
22. Hammer, N. D., et al. The curli nucleator protein, CsgB, contains an amyloidogenic domain that directs CsgA polymerization. Proc Natl Acad Sci USA 104, 12494-12499 (2007).
23. Huang, J. et al. CHARMM36m: an improved force field for folded and intrinsically disordered proteins. Nat Methods 14, 71-73 (2017).
24. Huang, J. et al. Programmable and printable Bacillus subtilis biofilms as engineered living materials. Nat Chem Biol 15, 34-41 (2019).
25. Jimenez, E. M. et al. Genetically Modifying the Protein Matrix of Macroscopic Living Materials to Control Their Structure and Rheological Properties. ACS Synth Biol 13, 3936-3947 (2024).
26. Jumper, J. et al. Highly accurate protein structure prediction with AlphaFold. Nature 596, 583-589 (2021).
27. Kang, S. Y. et al. Engineering Bacillus subtilis for the formation of a durable living biocomposite material. Nat Commun 12, 7133 (2021).
28. Kaveh-Baghbaderani, Y., et al. Magnetic Separation of Antibodies with High Binding Capacity by Site-Directed Immobilization of Protein A-Domains to Bare Iron Oxide Nanoparticles. ACS Applied Nano Materials 4, 4956-4963 (2021).
29. Ke, P. C. et al. Half a century of amyloids: past, present and future. Chem Soc Rev 49, 5473-5509 (2020).
30. Manjula-Basavanna, A., et al. Mechanically Tunable, Compostable, Healable and Scalable Engineered Living Materials. Nat Commun 15, 9179 (2024).
31. Manjula-Basavanna, A., et al. Robust Self-Regeneratable Stiff Living Materials Fabricated from Microbial Cells. Adv Funct Mater 31 (2021).
32. Milner-White, J. E., et al. Amyloid formation may involve alpha—to beta sheet interconversion via peptide plane flipping. Structure 14, 1369-1376 (2006).
33. Molinari, S. et al. A de novo matrix for macroscopic living materials from bacteria. Nat Commun 13, 5544 (2022).
34. Moser, F., et al. Light-Controlled, High-Resolution Patterning of Living Engineered Bacteria Onto Textiles, Ceramics, and Plastic. Advanced Functional Materials 29, 1901788 (2019).
35. Nguyen, P. Q., et al. Engineered Living Materials: Prospects and Challenges for Using Biological Systems to Direct the Assembly of Smart Materials. Advanced Materials 30, 1704847 (2018).
36. Nguyen, P. Q., et al. Programmable biofilm-based materials from engineered curli nanofibres. Nat Commun 5, 4945 (2014).
37. Phillips, J. C. et al. Scalable molecular dynamics with NAMD. J Comput Chem 26, 1781-1802 (2005).
38. Praveschotinunt, P. et al. Engineered E. coli Nissle 1917 for the delivery of matrix-tethered therapeutic domains to the gut. Nat Commun 10, 5580 (2019).
39. Rodrigo-Navarro, A., et al. Engineered living biomaterials. Nature Reviews Materials 6, 1175-1190 (2021).
40. Rosignoli, S. et al. Boosting the Full Potential of PyMOL with Structural Biology Plugins. Biomolecules 12 (2022).
41. Sercinoglu, O. et al. gRINN: a tool for calculation of residue interaction energies and protein energy network analysis of molecular dynamics simulations. Nucleic Acids Res 46, W554-W562 (2018).
42. Shea, D. et al. alpha-Sheet secondary structure in amyloid beta-peptide drives aggregation and toxicity in Alzheimer's disease. Proc Natl Acad Sci USA 116, 8895-8900 (2019).
43. Siri, M., et al. Curli Amyloid Fibers in Escherichia coli Biofilms: The Influence of Water Availability on their Structure and Functional Properties. Macromolecular Bioscience 24, 2300234 (2024).
44. Skanberg, R., et al. VIAMD: a Software for Visual Interactive Analysis of Molecular Dynamics. J Chem Inf Model 63, 7382-7391 (2023).
45. Sleutel, M., et al. Structural analysis and architectural principles of the bacterial amyloid curli. Nat Commun 14, 2822 (2023).
46. Smith, P., et al. On the interaction of hyaluronic acid with synovial fluid lipid membranes. Phys Chem Chem Phys 21, 9845-9857 (2019).
47. Tang, T.-C. et al. Materials design by synthetic biology. Nature Reviews Materials 6, 332-350 (2020).
48. Van Gerven, N., et al. Bacterial amyloid formation: structural insights into curli biogensis. Trends Microbiol 23, 693-706 (2015).
49. Zhang, C. et al. Engineered Bacillus subtilis biofilms as living glues. Materials Today 28, 40-48 (2019).
50. Zhong, C. et al. Strong underwater adhesives made by self-assembling multi-protein nanofibres. Nature Nanotechnology 9, 858-866 (2014).

What is claimed is:

1. A platform for producing autogenic engineered living materials (ELM), the platform comprising a plurality of engineered cells, wherein each engineered cell of the plurality expresses at least one non-native β-solenoid protein monomer, wherein the at least one β-solenoid protein monomer comprises a CsgA analog from a non model organism.

2. The platform of claim 1, wherein the at least one β-solenoid protein monomer expressed by each engineered cell of the plurality is the same.

3. The platform of claim 1, wherein the plurality of engineered cells comprises two or more sub-populations of engineered cells, wherein each of the two or more sub-populations expresses a different β-solenoid protein monomer.

4. The platform of claim 1, wherein the engineered cell comprises Bacillus subtilis, Saccharomyces cerevisiae, Pichia pastoris, Pseudomonas putida, or E. coli.

5. The platform of claim 1, wherein the engineered cell has been modified to remove a native curli operon.

6. The platform of claim 1, wherein the non-model organism comprises Halomonas saliphila, Alteromonas macleodii, Blastomonas sp. CACIA14H2, Erythrobacter longus, Ensifer sp. Root31, or any combination thereof.

7. The platform of claim 1, wherein the β-solenoid protein monomer is engineered to include one or more native E. coli sequences to facilitate extracellular secretion, self-assembly, or both, wherein the one or more native E. coli sequences comprise an N-terminal signal sequence (Sec), an N-terminal curli-specific targeting sequence (N22), or both Sec and N22.

8. The platform of claim 1, wherein the β-solenoid protein monomer comprises a plurality of cross-β repeat units, wherein each cross-β repeat unit comprises:
a first β-strand having a sequence KR₁-Ω-Ψ-Ω-Ψ-Ω-KR₇;
an intra-repeat loop having a sequence X-G-X-G,
a second β-strand having a sequence KR₁-Ω-Ψ-Ω-Ψ-Ω-KR₇; and
an inter-repeat loop having a sequence X-X-X-X;
wherein X represents a variable amino acid,
wherein KR₁ is selected from serine and asparagine;
wherein KR₇ is glutamine;
wherein Ω represents an outward-facing hydrophilic amino acid;
wherein Ψ represents an inward-facing hydrophobic amino acid; and
wherein each Ψ is independently selected from alanine, isoleucine, valine, leucine, phenylalanine, serine, and threonine.

9. The platform of claim 8, wherein the β-solenoid protein monomer comprises from about 10 to about 50 cross-β repeat units.

10. The platform of claim 1, wherein the β-solenoid protein monomer is engineered to contain at least one additional functional moiety.

11. The platform of claim 10, wherein the at least one additional functional moiety comprises an iron binding peptide or an immunoglobulin G (IgG) binding protein.

12. The platform of claim 10, wherein the at least one additional functional moiety is connected to the β-solenoid protein monomer via a flexible amino acid linker.

13. A method for producing β-solenoid protein monomers, the method comprising culturing the platform of claim 1.

14. A plurality of β-solenoid protein monomers produced by the method of claim 13.

15. The plurality of β-solenoid protein monomers of claim 14, wherein each individual β-solenoid protein monomer of the plurality comprises at least one additional functional moiety, wherein the at least one additional functional moiety comprises an iron binding protein or an IgG antibody binding protein.

16. An amyloid nanofiber comprising the plurality of β-solenoid protein monomers of claim 14.

17. A hydrogel comprising the amyloid nanofiber of claim 16.

18. The hydrogel of claim 17, wherein the hydrogel exhibits shear-thinning behavior.

19. An aquaplastic comprising the hydrogel of claim 17.

20. A method for 3D printing, the method comprising extruding the hydrogel of claim 17 through a nozzle using a layer-by-layer approach to build a three-dimensional article.
